(19) United States
(12) Patent Application Publication
(10) Pub. No.: US 2005/0176083 A1
(43) Pub. Date: Aug. 11, 2005

(54) Recombinant protein c variants

(76) Inventor: Bjorn Dahlback, Malmo (SE)

Correspondence Address: BIRCH STEWART KOLASCH & BIRCH, PO BOX 747, FALLS CHURCH, VA, 22040-0747, US

(21) Appl. No.: 10/506,080

(22) PCT Filed: Feb. 28, 2003
(86) PCT No.: PCT/SE03/00331

(30) Foreign Application Priority Data
Mar. 1, 2002 (US) 60360181
Aug. 6, 2002 (US) 60401042

(51) Int. Cl. C12Q 1/56; C07H 21/04; C12P 21/04; A61K 38/46; C12N 9/64; C12N 5/08

(52) U.S. Cl. 435/13; 435/69.6; 435/226; 435/320.1; 435/372; 536/23.2; 424/94.65

(57) ABSTRACT

The present invention is concerned with a variant blood coagulation component, which is substantially homologous in amino acid sequence to a wild-type blood coagulation component capable of exhibiting anticoagulant activity in the protein C-anticoagulant system of blood and selected from protein C (PC) and activated protein C (APC), said variant component being capable of exhibiting an anticoagulant activity, that is enhanced in comparison with the anticoagulant activity expressed by the corresponding wild-type blood coagulation component, and said variant component differing from the respective wild-type component, in that it contains in comparison with said wild-type component at least one amino acid residue modification in its N-terminal amino acid residue sequence that constitutes the Gla-domain of protein C and at least one amino acid residue modification in the serine-protease domain of protein C. The present invention is also concerned with methods to produce such variants based on DNA technology; with DNA segments intended for use in the said methods; and with use of said variants for therapeutic and diagnostic purposes.

### FIELD OF THE INVENTION

The present invention is directed to functional recombinant protein C variants that exhibit enhanced anticoagulant activity, and to use of such variants for therapeutic or diagnostic purposes. More specifically, the present invention is directed to protein C variants containing both a modified Gla-domain and a modified serine protease (SP) domain, and to use of such variants for therapeutic or diagnostic purposes.

### BACKGROUND OF THE INVENTION

Protein C is a vitamin K-dependent protein of major physiological importance that participates in an anticoagulant system of the blood, which is generally designated the protein C-anticoagulant system. Like all vitamin K-dependent proteins, protein C contains a Gla-domain or Gla-module that is comprised of the N-terminal 45 amino acid residues, said domain being crucial for membrane binding-affinity as will be discussed in more detail below. The SP-domain of protein C is involved i. a. in proteolytic activity and serpin resistance of protein C.

In said protein C-anticoagulant system, protein C functions in concert with other proteins including the cofactors protein S and intact Factor V (FV), which act as synergistic cofactors to protein C in its activated form (APC, Activated Protein C), as a down-regulator of blood coagulation, thereby preventing excess coagulation of blood and, thus, inhibiting thrombosis. This anticoagulant activity that is exhibited by the activated form of protein C emanates from its capacity to inhibit the reactions of blood coagulation by specifically cleaving and degrading activated Factor VIII (FVIIIa) and activated Factor V (FVa), these being other cofactors of the blood coagulation system. As a result thereof, activation of components necessary for blood coagulation, viz. Factor X (FX) and prothrombin, is inhibited and the activity of the coagulation system is dampered. Protein C is, thus, of major physiological importance for a properly functioning blood coagulation system.

The importance of protein C can be deduced from clinical observations. For instance, severe thromboembolism affects individuals with homozygous protein C deficiency and affected individuals develop thrombosis already in their neonatal life. The resulting clinical condition, purpura fulminans, is usually fatal unless the condition is treated with protein C. On the other hand, heterozygous protein C deficiency is associated with a less severe thromboembolic phenotype and constitutes only a relatively mild risk factor for venous thrombosis. It has been estimated that carriers of this genetic trait have a 5- to 10-fold higher risk of thrombosis as compared to individuals with normal protein C levels. More importantly, however, the most common genetic defect associated with thrombosis is also affecting the protein C system. This condition is usually referred to as APC resistance and is most frequently caused by a single point mutation in the FV-gene, which mutation leads to replacement of the amino acid residue Arg506 with a Gln residue in the FV amino acid sequence. Arg506 constitutes one of three cleavage sites in activated FV (FVa), which are sensitive to cleavage action by APC, and such mutated FVa is less efficiently degraded by APC than normal FVa (Dahlbäck, J. Clin. Invest. 1994, 94: 923-927). This mutated FVa is also designated R506QFVa, FVa Leiden and Q506 mutant FVa.

The physiological importance of protein C and activated protein C (APC) as anticoagulant components in the blood coagulation system indicates potential use of these substances for therapeutic purposes.

Indeed, protein C and its activated form APC have already been used to some extent for therapeutic purposes (Verstraete and Zoldholyi, Drugs 1995, 49: 856-884; Esmon et al, Dev. Biol. Stand. 1987, 67: 51-57; Okajima et al, Am. J. Hematol. 1990, 33: 277-278; Dreyfys et al, N. Engl. J. Med. 1991, 325: 1565-1568). More specifically, protein C purified from human plasma has been used as replacement therapy in homozygous protein C deficiency (Marlar and Neumann, Semin. Thromb. Haemostas. 1990, 16: 299-309) and has also been used successfully in cases with severe disseminated intravascular coagulation due to meningococcemia (Rivard et al, J. Pediatr. 1995, 126: 646-652). Moreover, in a baboon model of septicaemia (using E. coli), APC was shown to have a protective effect, which was particularly pronounced when the APC was given prior to the E. coli infusion (Taylor et al, J. Clin. Invest. 1987, 79: 918-925). In any event, the results obtained to date suggest that protein C may become a useful drug, not only for treatment of the above conditions but also for many other conditions, in which the coagulation system is activated, e.g. for the prevention and treatment of venous thrombosis, vascular occlusion after recanalization of coronary vessel after myocardial infarction (MI) and after angioplasty.

It is envisioned that therapeutic treatment of various conditions related to blood coagulation disturbances could be improved if variants of protein C having improved anticoagulant properties were available. Moreover, such variants would be useful as reagents to improve various biological assays for other components of the protein C system in order to obtain assays having improved performance.

The development of recombinant DNA technology in the past decades has had a tremendous impact on the possibilities to produce desired biological substances efficiently and/or to create biological substances having desired and optionally specifically designed properties. Indeed, not only functional variants of protein C but also essentially wild-type protein C have been produced by recombinant technology, e.g. as reported in the following references.

In U.S. Pat. No. 4,775,624 (Bang et at) recombinant production of human protein C derivatives is disclosed. However, only production of protein C polypeptides having functional activities essentially corresponding to human wild-type protein C is disclosed. Recently, wild-type protein C produced in accordance with this reference has been used successfully in treatment of severe sepsis (Bernard, G. R. et al., “Efficacy and Safety of Recombinant Human Activated Protein C for Severe Sepsis”, New England Journal of Medicine, Mar. 8, 2001; 344 (10): 699-709.

Use of protein C prepared by recombinant technique has also been disclosed in Berg et al, Biotechnique, 1993, 14: 972-978; and Hoyer et al, Vox Sang. 1994, 67: Suppl. 3: 217-220).

Functional variants of protein C obtained by mutagenesis directed to the activation peptide region, which includes residues 158-169, may have enhanced sensitivity to thrombin, such variants being activated by thrombin faster than wild-type protein C (Erlich et al, Embo. J. 1990, 9: 2367-2373; and Richardson et al., Nature 1992, 360: 261-264). In one of these studies (Richardson et al., Nature 1992, 360: 261-264), a number of mutations were introduced around the activation site leading to a mutant protein C, that was relatively easily activated by thrombin formed during coagulation of blood, even in absence of thrombo-modulin, which is a membrane protein that is usually required for efficient activation of protein C by thrombin.

More specifically, those protein C variants having enhanced interaction with thrombin that are disclosed in Richardson et al., Nature, 1992, 360: 261-264, comprise mutations in the activation peptide region, two putative inhibitory acidic residues near the thrombin cleavage site being altered. One protein C variant comprising said altered residues in the activation peptide region and also the Asn313Gln mutation disclosed by Grinnell et al. (infra) has recently been shown to function well as an anticoagulant in experiments performed in vivo (Kurz et al., Blood, 1997, 89: 534-540). However, in this protein C variant the enhanced anticoagulant activity is due to the Asn 313 Gin mutation, the other mutations giving rise to enhanced interaction with thrombin.

In Grinnell et al., J. Biol. Chem., 1991, 9778-9785, the role of glycosylation in the function of human protein C is examined, site-directed mutagenesis being used to singly eliminate each of the four potential N-linked glycosylation sites, i.e. the positions 97, 248, 313, and 329. In the protein C variants disclosed therein, Gln is substituted for Asn at positions 97, 248, and 313, resp., and it is shown, that the protein C mutants having this substitution mutation at positions 248 and 313 exhibit a 2- to 3-fold enhanced anticoagulant activity in addition to other modified properties.

Functional variants of protein C and APC that exhibit enhanced anticoagulant activity due to introduction of at least one amino acid residue modification in the amino acid sequence of wild-type protein C, e.g. in the serine protease (SP) module, which modification does not alter the glycosylation of protein C, are disclosed in WO 98/44000. One variant specifically disclosed therein contains a few mutations in the SP module that are located within a short amino acid residue stretch between the residue nos. 300 and 314, said variant exhibiting approximately 200% enhanced anticoagulant activity as compared to wild-type human protein C.

In J. Biol. Chem. 1993, 268: 19943-19948, Rezaie et al. disclose a protein C mutant comprising a Glu357Gln mutation (i.e. Glu192Gln if chymotrypsin numbering is used). This mutant inactivates FVa at an about 2- to 3-fold enhanced rate in a pure system, whereas in plasma, the anticoagulant activity is not enhanced as compared to wild-type protein C since the mutant is rapidly inhibited by protease inhibitors such as alpha 1-antitrypsin and antithrombin III.

Protein C variants having modifications in or lacking the Gla-domain of native protein C have also been reported previously.

For instance, a protein C variant lacking the Gla-domain of native protein C and comprising a Thr254Tyr mutation (i.e. Thr99Tyr based on the chymotrypsin numbering) is disclosed in J. Biol. Chem., 1996, 271: 23807-23814. This variant protein C has a 2-fold enhanced activity towards pure FVa, i.e. soluble FVa in absence of phospholipids, but is lacking anticoagulant activity in plasma by virtue of the missing Gla-domain.

Recently, a few protein C variants having a modified Gla-domain have been reported by Shen et al. in J. Biol. Chem., Vol. 273, No. 47, pp. 31086-31091, 1998. These protein C variants contain a few substitutions in the Gla-domain and exhibit enhanced Ca and/or membrane binding properties and, thus, also enhanced anticoagulant activity of activated protein C (APC). Some of these variants have also been disclosed in WO 99/20767 together with other protein C variants containing substitution modifications in the Gla-domain. The latter reference is generally related to modified vitamin K-dependent polypeptides exhibiting altered, e.g. enhanced, membrane binding-affinity due to modifications, i.e. substitutions, in their Gla-domains. The vitamin K-dependent polypeptide could comprise factor VII or any other vitamin K-dependent protein, e.g. protein C. It is to be noted that the numbering of the Gla-domain residues differs between Shen et al. and this WO reference in that according to the WO reference, position 4 in the protein C sequence is not occupied by any residue, which means that e.g. position 10 according to Shen (and the present invention) corresponds to position 11 according to the WO reference.

WO 01/59084 is concerned with human protein C derivatives that have retained important biologic activities as compared to wild-type protein C but have increased anticoagulant activity, resistance to serpin inactivation and increased sensitivity to thrombin when compared to wild-type protein C. These protein C derivatives contain an Asp167Phe substitution (D167F), an Asp172Lys substitution (D172K) and at least one further substitution specifically defined and contained in the Gla-domain or the SP-domain. Although a substitution of Y302Q or Y302E (i.e. in the SP-domain) is disclosed therein, no improved properties are verified with test data. Moreover, this substitution is envisioned to provide resistance to serpins but not to provide a truly enhanced anticoagulant activity, i.e. an anticoagulant activity that is enhanced per molecule but not necessarily over time.

In WO 01/36462, protein C variants are disclosed that contain a modified Gla-domain wherein one or more site-directed mutations have been performed at amino acid positions 10, 11 and 12 (His, Ser, Ser), viz. at amino acid 12, at amino acids 12 and 11, or at amino acids 12, 11 and 10, with an aim to replace Ser12 (phosphorytable) with a non-phosphorytable amino acid residue. Experimental results are only disclosed for a few variants and anticoagulant activity is only assessed as prolongation of clotting time in an activated partial thromboplastin time assay.

Even though protein C variants having enhanced anticoagulant activity and/or other modified properties have been disclosed previously, there is still a need of protein C variants that exhibit enhanced anticoagulant activity and/or have other beneficial properties that would be useful for therapeutic and/or diagnostic purposes.

Moreover, protein C variants containing both a modified Gla-domain and a modified SP-domain, which variants exhibit enhanced membrane binding affinities in addition to an anticoagulant activity that is enhanced per molecule but not necessarily prolonged over time, have not been reported earlier. Such variants could offer advantages, such as lower dose requirements or less frequent administration and/or quick on-set of anticoagulant activity, e.g. as compared to wild-type protein C. It is to be noted that the above-mentioned WO 01/59084 refers to protein C derivatives that have enhanced resistance to serpins and, thus, in addition to other improved properties have a prolonged, but not an enhanced, anticoagulant activity.

### SUMMARY OF THE INVENTION

The present invention is concerned with functional variants of protein C, that contain a modified Gla-domain and a modified SP-domain, which variants when activated exhibit enhanced anti-coagulant activity that preferably is enhanced per molecule. This enhanced anticoagulant activity of the present protein C variants emanates essentially from enhanced calcium and/or membrane binding properties due to the modified Gla-domain or an enhanced proteolytic, suitably amidolytic, activity due to the modified SP-domain or preferably both. Moreover, said activity is mainly expressed by APC, which is the active form of the protein C zymogen, said zymogen being virtually inactive. Accordingly, the present invention is also concerned with variants of APC that contain a modified Gla-domain and a modified SP-domain and exhibit enhanced anticoagulant activity. The Gla-domain comprises the first amino-terminal 45 residues of protein C and its structure and function will be discussed in more detail below. The SP-domain that in protein C from humans comprises 262 amino acid residues (nos. 158-419) is also discussed in more detail below.

According to the present invention it has been discovered that introduction of at least one, but preferably more than one amino acid residue modification into each of the Gla- and SP-domains, suitably at least 4, and specifically 7 or more modifications into the Gla-domain and 6 or more modifications into the SP-domain, provides protein C or APC variants that have improved properties, and specifically variants that have improved anticoagulant activity (per se or when activated), as compared to the wild-type protein.

Suitably, the present variants do not contain more than 10 amino acid modifications in their Gla-domain and not more than 10 amino acid modifications in their SP-domain and, preferably, do not encompass hybrids between different vitamin K-dependent proteins, such as hybrid protein C variants having a Gla-domain derived from prothrombin or Factor X, unless the differences between this other Gla-domain and the Gla-domain of protein C only constitute a few amino acid residues, so that the hybrid has a high degree of homology with wild-type protein C. Likewise, hybrids wherein the SP-domain and the remainder of protein C are derived from different species are usually not encompassed by the instant invention.

Protein C variants according to the present invention that display improved properties, such as much enhanced anticoagulant activity, could provide benefits, e.g. by lowering the dosage or frequency of administration when used for therapeutic purposes.

The present invention is also concerned with methods to produce such variants based on DNA technology, with DNA segments intended for use in said methods, and with use of said variants for therapeutic and/or diagnostic purposes.

In accordance with the present invention, anticoagulant activity that is enhanced as compared to the anticoagulant activity of the wild-type substance means an activity that is enhanced per molecule but not necessarily prolonged over time, e.g. due to a stabilized molecule.

### BRIEF DESCRIPTION OF THE DRAWINGS

In the following, the present invention is disclosed in more detail with reference to the drawings, wherein:

FIG. 1-5 are concerned with variants having mutations only in the SP-domain, viz.:

FIG. 1 illustrates the amidolytic activity of human and bovine wild-type APC and of APC mutants. Human APC (∘), human APC-SP (●), bovine APC (□), bovine APC-SP (▪).

FIG. 2A-C illustrate the effect of various APCs on the activated partial thrombo-plastin times in human and bovine plasma. A) In human plasma: human APC (∘), human APC-SP (●), bovine APC (□), bovine APC-SP (▪). B) In human plasma supplemented with bovine protein S (final concentration of 5 μg/ml): human APC (∘), human APC-SP (●), bovine APC (□), bovine APC-SP (▪). C) In bovine plasma: human APC (∘), human APC-SP (●), bovine APC (□), bovine APC-SP (▪).

FIG. 3A-C illustrate the effect of various APCs on the inactivation of human factor VIIIa. Different concentrations of various APCs were preincubated with factor VIIIa, factor IXa, phospholipids and Ca²⁺ mixture for 5 min in the presence of bovine factor V and human or bovine protein S. Factor X was activated by this solution and the rate of factor Xa formation was measured with a synthetic substrate. The absorbence was linearly related to the factor VIIIa activity, and results were expressed as percentage of respective control. A) Inactivation of factor VIIIa by high concentrations of APCs (final concentrations are indicated) in the presence of human protein S and bovine factor V: human APC (∘), human APC-SP (●), bovine APC (□), bovine APC-SP (▪). B) Inactivation of factor VIIIa by low concentrations of APCs (final concentrations are indicated) in the presence of human protein S och bovine factor V: human APC (∘), human APC-SP (●), bovine APC (□), bovine APC-SP (▪). C) Inactivation of factor VIIIa by APCs (final concentrations are indicated) in the presence of bovine protein S and bovine factor V: human APC (∘), human APC-SP (●), bovine APC (□), bovine APC-SP (▪).

FIGS. 4A and B illustrate the effect of various APCs on the prothrombin times in human and bovine plasma. A) In human plasma: human APC (∘), human APC-SP (●), bovine APC (□), bovine APC-SP (▪). B) In bovine plasma: human APC (∘), human APC-SP (●), bovine APC (□), bovine APC-SP (▪).

FIG. 5 illustrates the inactivation of various APCs, viz. human APC (∘), human APC-SP (●), bovine APC (□) and bovine APC-SP (▪), by human plasma.

FIG. 6-14 are concerned with variants having mutations in the Gla-domain, viz.:

FIG. 6 illustrates the effect of various APC variants (mutants) on the activated partial thromboplastin times (APTT) in human plasma. The following APC variants were examined: human wild-type (wt) APC (●), APC mutant QGN (□), APC mutant QGED (▴), APC mutant GNED (x), APC mutant SEDY (|) and APC mutant ALL (or QGNSEDY) (Δ).

FIG. 7 illustrates impact of human protein S on the effect of APC (wt and mutant) in an APTT assay. The following APC variants were examined: wt APC (●) and APC mutant QGNSEDY (ALL) (□).

FIG. 8 illustrates the effect of various APC variants on the prothrombin times in human plasma. The following APC variants were examined: wt APC (●), APC mutant QGN (□), APC mutant QGED (▴), APC mutant GNED (x), APC mutant SEDY (|), and APC mutant QGNSEDY (ALL) (Δ).

FIG. 9 illustrates the capacity of various APC variants to inactivate human factor Va as measured by the thrombin generation due to FXa-mediated activation of prothrombin, said activation being potentiated by FVa. The following APC variants were examined: wt APC (●), APC mutant QGN (□), APC mutant SEDY (+), and APC mutant QGNSEDY (ALL) (Δ).

FIG. 10 illustrates the capacity of various APC variants to inactivate human factor Va, the activity of FVa being measured with a prothrombinase assay. The following APC variants were examined: wt APC (●), APC mutant QGN (▴), APC mutant SEDY (Δ), and APC mutant QGNSEDY (ALL) (□).

FIG. 11 illustrates inactivation of normal, i.e. wild-type (wt), FVa and Q506 mutant FVa (FVa Leiden) by APC. Values are shown for inactivation of: wt FVa with wt APC (●); wt FVa with APC mutant QGNSEDY (ALL) (□); R506Q FVa with wt APC (▴); and R506Q FVa with APC mutant QGNSEDY (ALL) (x).

FIG. 12-14 illustrate the ability of wt and variant protein C to bind to phospho-membranes. A surface plasma resonance technique from BIAcore was used. In these figures, different phospholipids were used, viz. 100% phosphatidylcholine (FIG. 12); a mixture of 20% phosphatidylserine and 80% phosphatidylcholine (FIG. 13); and a mixture of 20% phosphatidylserine, 20% phosphatidylethanolamine and 60% phosphatidylcholine (FIG. 14). In all tests, human wild-type protein C (wt) and the APC variants QGNSEDY (ALL), SEDY, SED, and QGN, were analyzed.

FIG. 15-25 are concerned with variants according to the present invention, i.e. variants that contain mutation(s) both in the SP-domain and in the Gla-domain, viz.:

FIG. 15 illustrates the effect of a mutant (super-Apc) comprising the mutated Gla-domain of QGNSEY (ALL) and a mutated SP-domain on the activated partial thromboplastin time (APTT) in human plasma.

FIG. 16 illustrates effects of recombinant APC variants in an APTT reaction.

FIG. 17 illustrates the effect of GNED-SP on tissue factor induced clotting.

FIG. 18 illustrates effects of APC variants in an APTT reaction.

FIG. 19 illustrates effects of APC variants on TF-induced clotting.

FIG. 20 illustrates effects of APC variants on APTT clotting time in presence of Mab HPS54 (protein S specific).

FIG. 21 illustrates effects of APC variants on whole blood clotting.

FIG. 22 illustrates effects of APC variants in an APTT reaction using rat plasma.

FIG. 23 illustrates effects of APC variants in TF-induced clotting using rat plasma.

FIG. 24 illustrates effects of APC variants in a mouse APTT reaction.

FIG. 25 illustrates effects of APC variants on tissue-factor induced clotting of mouse plasma.

### DETAILED DESCRIPTION OF THE INVENTION

A. Molecular arrangement of Protein C The protein C molecule is composed of four different types of modules or domains. In the direction of amino terminus to carboxy terminus, these consist of a Gla-module, two EGF-like modules, i.e. Epidermal Growth Factor homologous modules, and finally a typical serine protease (SP) module. In plasma, most of the circulating protein C consists of the mature two-chain, disulfide-linked protein C zymogen arisen from a single-chain precursor by limited proteolysis. These two chains are the 20 kDa light chain, which contains the Gla- and EGF-modules and the 40 kDa heavy chain, which constitutes the SP-module. During activation by thrombin bound to thrombomodulin, a peptide bond Arg-Leu (residues 169 and 170) is cleaved in the N-terminal part of the heavy chain and an activation peptide comprising twelve amino acid residues (residues 158-169) is released. In connection with the present invention, the numbering of residues in the amino acid sequence of protein C and variants thereof is based on mature protein C.

The amino acid sequence of protein C has been deduced from the corresponding cDNA-nucleotide sequence and has been reported in the literature. Morover, the cDNA-nucleotide sequences and the corresponding amino acid sequences for protein C are available from the EMBL Gene database (Heidelberg, Germany) under the accession number X02750 for human protein C, which is designated HSPROTC, and the accession number KO 2435 for bovine protein C, which is designated BTPBC.

As stated above, the Gla-domain of the vitamin K-dependent proteins comprises the N-terminal 45 amino acid residues. Thus, the amino acid sequence of the entire Gla-domain is known for proteins, such as human and bovine protein C, for which the entire amino acid sequence or the N-terminal part thereof (45 residues) has been determined. Based on the above database sequences, the Gla-domain of human protein C and bovine protein C can be illustrated as shown below (SEQ ID NO:1 and SEQ ID NO:2, respectively):

(SEQ ID NO:1)

ANSFLEELRH SSLERECIEE ICDFEEAKEI FQNVDDTLAF WSKHV

(SEQ ID NO:2)

ANSFLEELRP GNVERECSEE VCEFEEAREI FQNTEDTMAF WSKYS

Likewise, the amino acid sequences of the SP-domains (human and bovine, respectively) may be obtained from these database sequences, wherein the SP-domain of human protein C is comprised of amino acid residue nos. 158-419 and the bovine SP-domain is comprised of amino acid residues 158-417. Preferably, the modifications in the SP-domain are located in an amino acid residue stretch between and inclusive of amino acid nos. 290 and 320 of the human SP-domain, said stretch corresponding to the following amino acid sequence:

| | |
|---|---|
| QAGQETLVTG WGYHSSREKE AKRNRTFVLN F | (SEQ ID NO:3) |

In the SP-domain of bovine protein C, a corresponding, but shorter, amino acid stretch between and inclusive of amino acid nos. 292 and 318 has the following amino acid sequence:

| | |
|---|---|
| QVGQETVVTGW GYRDETKRNR TFVLSF | (SEQ ID NO:4) |

In connection with the selection of modification targets in the Gla-domain, a comparison of such N-terminal sequences as regards similarities as well as deviations between individual sequences (from different vitamin K-dependent proteins and/or from different species) could indicate positions in the Gla-domain of protein C that could be suitable as mutagenesis (i.e. modification) targets. For such a comparison it may not be necessary to know the entire amino acid sequence of the Gla-domain but it could be sufficient if the amino acid residues at positions potentially important for anticoagulant activity have been determined. A similar comparison of SP-domains in protein C of different species, e.g. between human and bovine SP-domains, or specific partial sequences thereof, may indicate positions in the SP-domain that could be suitable mutagenesis targets.

In connection with the present invention, the usual 1-letter or 3-letter symbols are used as abbreviations for amino acids as is shown in the following table of correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| J | Xaa | Unknown or other |

B. Variants of Protein C

As stated above, the present invention is concerned with functional variants of recombinant protein C, said variants containing a modified Gla-domain and a modified SP-domain, and said variants displaying enhanced anticoagulant activity. These variants differ from wild-type recombinant protein C as regards one or more, suitably a few and preferably 10-15, amino acid residues, said residues being inserted, deleted or substituted (i.e. replaced) both in the Gla-domain and in the SP-domain of the corresponding wild-type sequence, thereby giving rise to the present variants of protein C. Since said differences are maintained after activation of protein C to APC, the present invention is also concerned with APC variants having enhanced anticoagulant activity. According to a suitable embodiment of the present invention, modification(s) in the Gla-domain is (are) substitution(s) and the SP-domain contains at least one substitution and at least one deletion.

At present, such variants are conveniently obtained by mutagenesis, especially site-directed mutagenesis including use of oligonucleotide primers. However, the present invention is concerned with the functional variants per se irrespective of the mode of obtaining these variants.

In view of the close relationship between PC and APC, frequently, no clear distinction is made between PC and APC in connection with the present invention, but the designation PC/APC is used and the context will reveal if one or both of these substances are considered. Moreover, the protein C zymogen is virtually inactive and, thus, enhanced anticoagulant activity of protein C is essentially only exhibited after activation of said zymogen in vivo or in vitro. Accordingly, in context with the present invention, the expression “protein C variants that exhibit enhanced anticoagulant activity” or the like, means that this enhanced activity is exhibited after activation of the protein C (zymogen) variant or that said variant is an APC variant.

In connection with the present invention, the expression “variant” means a modified wild-type molecule, such as a mutant molecule, that generally has a high degree of homology, suitably at least 90% homology, as compared to the wild-type molecule.

Accordingly, such variants suitably encompass only a few modified amino acid residues, and possibly only one amino acid residue in each of the Gla- and SP-domains, in order to preserve substantial homology with respect to the wild-type substance. This is of particular importance in connection with use of the present variants for treatment in vivo to avoid, or at least reduce, a possible immune response to the variant used for treatment.

Thus, for pharmaceutical purposes, preferably, the present variants are substantially homologous to the corresponding wild-type substance and contain only point mutations, e.g. one or a few single amino acid residue substitutions, deletions and/or insertions in each of said domains. Preferably, the variants contain more than one amino acid residue modification in each of said domains and could for use in vivo contain as many as up to 10 or even more amino acid residue modifications in each of said domains.

Accordingly, suitable variants of PC/APC have a high degree, viz. at least 90%, suitably at least 95%, preferably at least 97%, and specifically at least 98%, of amino acid sequence identity with wild-type mature PC/APC.

In connection with the diagnostic embodiments of the present invention, a high degree of homology is of course of less importance, the main requirement being that the functional variant exhibits one or more of the desired activities at an enhanced level as compared to the wild-type protein.

For pharmaceutical purposes, preferred embodiments of the present invention are concerned with human PC/APC variants. However, the present invention is also concerned with other PC/APC variants of mammalian origin, e.g. of bovine origin or murine origin, such as variants of mouse or rat origin, that have enhanced membrane binding properties and enhanced anticoagulant activity due to a modified Gla-domain and a modified SP-domain.

As mentioned above, the Gla-domain or Gla-module is specific for the vitamin K-dependent protein family, the members of which contain a specific protein module (said Gla-module), wherein the glutamic acid (E) residues are modified to γ-carboxy glutamic acid residues (Gla). This modification is performed in the liver by enzymes that use vitamin K to carboxylate the side chains of the glutamic acid residues in the protein C precursor. In the sequences (SEQ ID NOS: 1 and 2), given above for the Gla-domain of human and bovine protein C, respectively, the E residues are thus converted to Gla-residues in the circulating protein.

The Gla-domain is comprised of the first amino-terminal 45 residues of the vitamin K-dependent protein and provides the protein with the ability to bind calcium and to bind negatively charged procoagulant phospholipids. Moreover, a membrane contact site, that is of crucial importance for the function of activated protein C (APC) in proteolysis of FVa and FVIIIa, is contained in said Gla-domain, the activity of APC being exhibited upon association of APC and other proteins, i.e. factor V and protein S cofactors, on a membrane surface. However, despite a high degree of sequence homology between the Gla-containing regions of different vitamin K-dependent proteins, these proteins display a large range of membrane affinities. This indicates that it could be possible to modify, and more specifically to enhance, membrane affinity of protein C, e.g. human protein C, which is a low affinity protein.

For this purpose, the structures of high affinity vitamin K-dependent proteins could serve as a template to suggest possible modifications that could enhance membrane binding-affinity and, thus, anticoagulant activity of low affinity proteins, such as protein C, as suggested by Shen et al. (supra). For instance, site-directed mutagenesis could be performed on wild-type protein C to produce protein C variants having a structure that approaches the structure of high affinity vitamin K-dependent proteins, such as protein Z.

However, although the existence of a common archetype for electrostatic distribution that would be valid for all vitamin K-dependent proteins and would predict possible positions for amino acid modifications that could give rise to enhanced membrane binding-affinity is suggested in the WO 99/20767 publication, this archetype is only concerned with a few positions of the Gla-domain, viz. 10, 11, 28, 32, and 33 (according to the numbering used in connection with the present invention). Moreover, as reported by Shen et al. (supra), protein C has been shown to have unique features and would not necessarily fit into such a common hypothesis.

The SP-domain of protein C contains sequences that interact with sequences in APC-inhibitors, e.g. α1AT (alpha 1-anti-trypsin) and PCI (protein C inhibitor), and also sequences that interact with sequences in FVa or FVIIIa. Such sequences of APC/PC constitute putative targets for site-directed mutagenesis performed in order to produce APC/PC variants that have an anticoagulant activity that is enhanced per molecule and optionally also are resistant to serpins and other APC-inhibitors.

In accordance with the present invention it has unexpectedly been found that modification(s) can be introduced into both the Gla-domain and the SP-domain of protein C to produce a variant protein C that exhibits improved properties in vivo and in vitro, such as enhanced membrane-binding affinity or enhanced anticoagulant activity and preferably both, while maintaining other desirable biological properties, such as fibrinolytic and anti-inflammatory activities.

B(1) Modifications in the Gla-Domain

In this section, suitable modifications in the Gla-domain are disclosed. The modified variants thereby obtained also contain at least one modification in their SP-domain as disclosed in B(2) further below.

The present variants contain in the Gla-domain at least one, suitably at least 4, e.g. 4-6 or 7-10, amino acid modification(s), such as substitutions (replacements), deletions or insertions (additions).

According to one aspect of the invention, in the Gla-domain said at least one amino acid modification is a substitution of one amino acid residue for another residue at any position of the Gla-domain of protein C. According to a further aspect of the invention, said position is a position other than positions 10, 11, 28, 32 or 33. According to a suitable embodiment of the present invention, said at least one amino acid modification is located at position 23, or 44.

A further aspect of the invention is concerned with protein C variants where said at least one amino acid modification is a substitution mutation selected from D23S and H44Y.

One embodiment of the present invention is concerned with protein C variants wherein said at least one amino acid modification is located at a position selected from the group consisting of amino acid residues nos. 1-9, 13-27, 29, 30, 31, and 34-45, or at a position selected from the group consisting of amino acid residues nos. 1-3,5-7, 9, 12-27, 29-31, and 36-45.

Other embodiments of the present invention are concerned with protein C variants, where said at least one amino acid modification in the Gla-domain is located at a position selected from amino acid residues nos. 10, 11, 12, 23, 32, 33 and 44. Suitably, more than one of, and preferably all, amino acid residues nos. 10, 11, 12, 23, 32, 33 and 44 are modified e.g. by substitution.

According to one aspect of the present invention, said at least one amino acid modification is comprised of one or more amino acid modifications other than the single modifications or the combination of modifications that are defined in the sequences E10G11E32D33, Q10G11E32D33, G11N12E32D33, G11E32D33, E32D33, and E32. According to common practice, e.g. E32D33 means a mutated sequence wherein at position 32, E has replaced the wild-type residue (O) and at position 33, D has replaced the wild-type residue (N). Alternatively, the present variants could contain one or more of these modifications (mentioned immediately above) in the Gla-domain and at least one, and suitably more than one, modification in the SP-domain. Optionally, such variants also contain at least one further modification in the Gla-domain, e.g. Y44.

A specific human protein C variant having much enhanced anticoagulant activity contains all of the substitution mutations H10Q, S11G, S12N, D23S, Q32E, N33D and H44Y. Thus, in addition to at least one modification in the SP-domain, this protein C variant has a modified Gla-domain having the following amino acid sequence:

| | |
|---|---|
| ANSFLEELRQ GNLERECIEE ICSFEEAKEI | (SEQ ID NO:5) |
| FEDVDDTLAF WSKYV | |

Another aspect of the present invention is concerned with protein C variants that contain one or more of the afore-said substitutions as the sole mutations in the Gla-domain, and suitably with a variant containing the substitutions S11G, S12N, Q32E, and N33D.

B(2) Modifications in the SP-Domain

According to the present invention one or more modifications in the Gla-domain is (are) combined with at least one modification in the SP-domain.

Except for the work of Grinnell et al. (supra), which is related to the role of glycosylation in the function of human PC, and for WO 98/44 000, there are no reports in the prior art literature, which indicate that one or more mutations in this module, i.e. the serine protease (SP) module, of the PC/APC molecule would lead to enhanced proteolytic and anticoagulant activities, that are enhanced per molecule. However, it was previously known that, on one hand, human APC is inhibited by several serpins, i.e. snake venom proteins, by the protein C inhibitor (PCI) and by alpha 1-anti-trypsin (α1AT), whereas, on the other hand, bovine APC is not inhibited by α1AT. In an effort to understand this phenomenon, Holly and Foster (Biochemistry, 1994, 33:1876-1880) constructed hybrid molecules between human and bovine protein C and were able to demonstrate that the molecular background for this difference resides somewhere in the SP-module of protein C. However, it is not suggested in or obvious from this report that mutations in the SP-module could lead to enhanced proteolytic and anticoagulant activities. Even though Holly and Foster actually did construe a PC variant that contains a modified SP-domain wherein amino acid residues nos. 300-314 are the same as in SEQ ID NO:6 disclosed below, they did not disclose any enhanced anti-coagulant activity of this variant, not even a prolonged anticoagulant activity.

The present Inventor has studied the SP-module in more detail in an attempt to locate closely the site in the SP-module, which is responsible for the different reactivities of human and bovine APC with α1AT. In connection with these studies, it was quite unexpectedly found that an amino acid sequence between (and inclusive of) residues numbers 300 and 314 in human wild-type protein C is essential for proteolytic and amidolytic activities and, thus, for the anticoagulant activity of PC/APC and that introduction of mutation(s) in this amino acid stretch could give rise to functional variants of PC/APC exhibiting said activities at higher rates as compared to the wild-type substance. This finding is the subject of WO 98/44 000 cited above.

Through continued scientific experiments, analysis, and innovation, the present Inventor has found that it is possible to combine modification(s) in the SP-domain and modification(s) in the Gla-domain to produce PC variants that contain mutations both in the Gla-domain and in the SP-domain and exhibit both enhanced membrane binding affinities and enhanced proteolytic and/or amidolytic activities and, thus, enhanced anticoagulant activity, while maintaining other desirable properties.

Thus, a suitable embodiment of the present invention is directed to functional variants of PC/APC, which express enhanced proteolytic and anticoagulant activities, which variants differ from the wild-type PC/APC in that they contain in addition to a modified Gla-domain as discussed above, also one or more mutations in their SP-module. In accordance with a specific embodiment, the present invention contemplates variants of PC/APC, wherein the mutation(s), suitably point mutation(s), in the SP-module is (are) located within an amino acid stretch consisting of the residues numbers 290-320, and suitably of the residues numbers 300-314 of wild-type human protein C.

In human PC/APC, the above mentioned sequence consisting of residues nos. 300-314 is comprised of the sequence WGYHSSREKE AKRNR (SEQ ID NO:6), the one letter code for amino acids being used. One preferred embodiment of the present invention is directed to a human PC/APC variant having an amino acid sequence identical with that of the wild-type PC/APC molecule except for mutation(s) contained in the Gla-domain and in said amino acid sequence (SEQ ID NO:6), the mutated sequence in the SP-domain being comprised of WGYRDETKRNR (SEQ ID NO:7).

The locations in the wild-type molecule of the mutations are obvious from the following representation of the mutated sequence: WGY . . . RD.ETKRNR (SEQ ID NO:7), wherein the points illustrate deleted amino acids and substitutions are underlined. Thus, the PC/APC variant of this specific embodiment contains an amino acid stretch in the SP module which is shortened with four amino acid residues and contains two substitutions in comparison with the wild-type PC/APC molecule.

A suitable embodiment of the present invention is, thus, concerned with PC/APC variants containing at least one modification in the Gla-domain and containing deletion and substitution mutations in the SP-module within the stretch consisting of amino acid residues 300-314. Preferably the amino acid residues nos. 303, 304, 305 and 308 are deleted and the amino acid residues nos. 307 and 310 are substituted (E307D/A310T) to produce the above mentioned PC/APC variant comprising the mutated sequence of SEQ ID NO:7. Accordingly, preferred variants containing mutations within the said sequence contain in the SP-domain a mutated sequence represented by the sequence of SEQ ID NO:7 instead of the wild-type sequence represented by the sequence of SEQ ID NO:6.

B(3) Modifications in the Gla-Domain and the SP-Domain

The present PC variants contain at least one modification in the Gla-domain and at least one modification in the SP-domain and, suitably, more than one modification in each domain. More specifically, the present invention is concerned with PC variants that have modifications in the Gla-domain as stated in B(1) and modifications in the SP-domain as stated in B(2). The present variants could contain these modifications in any combination. Moreover, specifically recited amino acid substitutions could be replaced with other substitutions that provide the same effect, i.e. another amino acid residue of like characteristics is used to replace the wild-type residue. Furthermore, deletion, addition or replacement mutations could be added, which mutations result in changes which do not affect the basic characteristics of the invention. Such modifications are evident from the discussion of mutagenesis strategy in the following section B(4). For instance, a substitution of a specific amino acid selected from group I as listed in B(4) for a wild-type amino acid residue could be replaced with a substitution of any other amino acid residue belonging to this group for said wild-type residue.

A suitable embodiment of the present invention is concerned with protein C variants having substitutions at positions 10, 11, 12, 23, 32, 33, and 44 in the Gla-domain and containing mutations in an amino acid stretch between and inclusive of positions 290 and 320, preferably between and inclusive of positions 300 and 314, and more specifically at positions 303, 304, 305, 307, 308, and 310 in the SP-domain. Other suitable protein C variants contain modifications within an amino acid stretch between and inclusive of positions 303 and 310, or within an amino acid stretch between and inclusive of positions 302 and 316. Suitable modifications in the SP-domain are deletions, optionally together with at least one substitution.

In accordance with one preferred embodiment, the protein C variant contains the substitutions H10Q, S11G, S 12N, D23S, Q32E, N33D and H44Y in the Gla-domain and deletions at positions 303, 304, 305, and 308 and the substitutions E307D and A310T in the SP-domain. Herein, said variant is frequently designated “super-APC”. According to a further embodiment, the protein C variant contains the same SP-domain mutations as super-APC but contains a Gla-domain containing only the substitutions S11G, S12N, Q32E and N33D.

Other mutations in the SP-domain and in the Gla-domain that could be combined to produce PC/APC variants having strongly enhanced anticoagulant activity are discussed below.

The mutations in the SP domain should be associated with at least slightly increased anticoagulant activity and optionally also enhanced amidolytic activity. The prime example is the SP mutant previously described herein (section B(2)). However, many other variants can be created by mutagenesis of this region in protein C, i.e. the 300-314 amino acid residues region, which comprises the so called 148 loop in the serine protease domain.

The mutations in the Gla domain can also be comprised of many different mutations but in principle they should by themselves result in enhanced or altered phospholipid-binding ability. Suitable mutations in the Gla domain, some of which have already been described herein, include E32; E32D33; G11; Q10G11; G11N12; Q10G11N12; S23; S23E32D33Y44, Q10G11E32D33, G11N12E32D33, Q10G11N12S23E32D33Y44 but many other variants are also possible. Positions of interest to mutate in the Gla-domain thus include Nos. 10, 11, 12, 23, 28, 32, 33, 34, 35 and 44.

In principle, any and all of the Gla-domain variants that have been discussed previously in this specification and in prior art and also such variants that further contain prior known mutations, e.g. the carbohydrate affecting mutations previously described by Grinnell et al. (supra) and/or mutations disclosed in WO01/59084 can be used together with the SP mutations disclosed in section B(2) and below.

One SP mutant specifically disclosed herein contains the sequence WGY . . . RD.ETKRNR. (SEQ ID NO:7) as compared to the wt human protein C sequence in this region, viz. WGYHSSREKEAKRNR (SEQ ID NO:6). Based on the idea that the loop should be shortened, a number of alternative mutations are listed below.

| | | | |
|---|---|---|---|
| | | Substitution | SEQ |
| Amino acid | Position of | Position/ | ID |
| sequence | deletion | residue | NO |

Single amino acid residue deletions:

| | | |
|---|---|---|
| WGY.SSREKEAKRNR | 303 | 8 |
| WGYH.SREKEAKRNR | 304 | 9 |
| WGYHS.REKEAKRNR | 305 | 10 |
| WGYHSS.EKEAKRNR | 306 | 11 |
| WGYHSSR.KEAKRNR | 307 | 12 |
| WGYHSSRE.EAKRNR | 308 | 13 |
| WGYHSSREK.AKRNR | 309 | 14 |

Double deletions:

| | | |
|---|---|---|
| WGY..SREKEAKRNR | 303, 304 | 15 |
| WGYH..REKEAKRNR | 304, 305 | 16 |
| WGYHS..EKEAKRNR | 305, 306 | 17 |
| WGYHSS..KEAKRNR | 306, 307 | 18 |
| WGYHSSR..EAKRNR | 307, 308 | 19 |
| WGYHSSRE..AKRNR | 308, 309 | 20 |

Triple deletions:

| | | |
|---|---|---|
| WGY...REKEAKRNR | 303, 304, 305 | 21 |
| WGYH...EKEAKRNR | 304, 305, 306 | 22 |
| WGYHS...KEAKRNR | 305, 306, 307 | 23 |
| WGYHSS...EAKRNR | 306, 307, 308 | 24 |
| WGYHSSR...AKRNR | 307, 308, 309 | 25 |

Other variations:

| | | | |
|---|---|---|---|
| WGY...RE.EAKRNR | 303, 304, 305, 308 | | 26 |
| WGY...RE.ETKRNR | 303, 304, 305, 308 | 310T | 27 |
| WGYH....REAKRNR | 304, 305, 306, 307 | 308R | 28 |
| WGY...KE.EAKRNR | 303, 304, 305, 308 | 306K | 29 |
| WGY...KD.EAKRNR | 303, 304, 305, 308 | 306K, 307D | 30 |
| WGY...KE.ETKRNR | 303, 304, 305, 308 | 306K, 310T | 31 |
| WGY...RQ.ETKRNR | 303, 304, 305, 308 | 307Q, 310T | 32 |
| WGY...RQ.EAKRNR | 303, 304, 305, 308 | 307Q | 33 |

Following this strategy there is quite a large number of variations that are possible. Theoretically, these could be found through modern molecular biology tools that allow random variation of the seven amino acid residues that are subject for deletions or replacements. The screening could build on the ability of the interesting mutants to yield enhanced catalytic activity against synthetic substrates.

Other positions in the SP-domain that could be modified are positions 302 and 316. At these positions the wt amino acid could be substituted with an amino acid selected from Ser, Ala, Thr, His, Leu, Lys, Arg, Asn, Asp, Glu, Gly, and Gln, e.g. the substitution being Y302Q or Y302E As stated above, the modified, i.e. variant or mutant, PC/APC of the present invention that contains at least one modification in the Gla-domain and at least one modification in the SP-domain has enhanced membrane binding affinities and enhanced proteolytic and/or amidolytic activities and, thus, enhanced anticoagulant activity. Such anticoagulant activity can be determined, i. a. as the ability of the present variants to increase clotting time in standard coagulation assays in vitro. The enhanced anticoagulant activity is measured in comparison to wild-type PC/APC which may be derived from plasma or obtained by recombinant DNA technique. Thus, to be useful in accordance with the present invention, the PC/APC variants should exhibit an anticoagulant activity, which is higher than the anticoagulant activity of the wild-type substance. Suitably, the present variants exhibit an anticoagulant activity which is enhanced at least about 50%, and suitably at least about 100%. Preferred PC/APC variants exhibit an anticoagulant activity that is enhanced about 400% or more, e.g. up to 1000% or even up to 3000% over wild-type protein C.

It is envisioned that mutagenesis in the Gla-domain, apart from enhanced membrane-binding also could provide other improved properties. For instance, since the Gla-domain has sites for interaction with some other proteins, the Gla-domain probably can interact with Protein S and factors V and VIII. Thus, it is envisioned that interaction with these proteins could be improved by mutations in the Gla-domain.

As stated above, the present variants preferably have a high degree of homology with the corresponding wild-type substance. Thus, the present variants preferably only contain point mutations. i.e. one or a few single amino acid residue substitutions, deletions and/or insertions.

Preferred embodiments of the present invention are concerned with human PC/APC variants. However, the present invention is also concerned with PC/APC variants of mammalian origin, e.g. bovine and murine, such as mouse and rat, origin, having enhanced anticoagulant activity.

According to another embodiment of the present invention, the variants could further contain one or a few mutations previously disclosed for protein C provided that these variants still exhibit an enhanced anticoagulant activity in comparison to the wild-type substance. Such mutations could be located in the Gla-domain, in the SP-domain and/or in other domains of the protein C molecule.

The present modifications may also be combined with an active-site modification in APC. The active site of APC may be inactivated by site-directed mutagenesis of the active site or chemically, for instance by N-dansyl-glutamyl-glycyl-arginyl-chloromethyl-ketone. Cf. Sorensen et al., 1997, J. Biol. Chem., 272:11863-11868. Since active-site modified APC is an inhibitor of the prothrombinase complex, active-site modified APC that exhibits enhanced membrane affinity may provide therapeutically advantageous APC variants.

B(4) Mutagenesis Strategy

To the man skilled in the art, it is evident that modifications in the Gla-domain other than substitutions and modifications other than deletions and substitutions in the SP-domain could provide protein C variants having properties that are improved as stated above. Moreover, other substitutions than those specifically mentioned herein could also provide such improved variants. Such substitutions could be conservative or non-conservative. Based on common side chain properties, naturally occurring residues are divided into the following classes:
1) hydrophobic residues comprising norleucine, Met, Ala, Val, Leu and lie;
2) neutral hydrophilic residues comprising Cys, Ser and Thr;
3) acidic residues comprising Asp and Glu;
4) basic residues comprising Asn, Gln, His, Lys and Arg;
5) residues that influence chain orientation comprising Gly and Pro; and
6) aromatic residues comprising Trp, Tyr and Phe.

Non-conservative substitutions may involve replacement of a member of one of these classes with a member of another class whereas conservative substitutions may involve replacement of an amino acid residue with a member of the same class. Positions of interest for substitutional mutagenesis include positions where the amino acid residues found in wild-type protein C from different species differ, e.g. as regards side-chain bulk, charge, and/or hydrophobicity. However, other positions of interest are such positions where the particular amino acid residue does not differ between, but are identical for, at least a few different species, since such positions are potentially important for biological activity. Initially, candidate positions are substituted in a relatively conservative manner. Then, if such substitutions result in a change of biological activity, more substantial substitutions are introduced and/or other modifications, such as additions, deletions or insertions, are made and the resulting variants screened for biological activity.

Since conservative substitutions or modifications of the amino acid sequence could be expected to produce variants having functional and chemical characteristics that are similar to those of wild-type protein C, suitably, the present protein C variants contain at least one non-conservative substitution, e.g. a substitution of an aromatic residue for a basic residue or a basic residue for an acidic residue.

Since the modified, i.e. variant or mutant, PC/APC of the present invention has enhanced anticoagulant activity, the above-mentioned screening for biological activity is suitably concerned with measurement of anticoagulant activity. Such anticoagulant activity can be determined i. a. as the ability of the present variants to increase clotting time in standard in vitro coagulation assays. The enhanced anticoagulant activity is measured in comparison to wild-type PC/APC, which may be derived from plasma or obtained by recombinant DNA technique. Thus, to be useful in accordance with the present invention, the PC/APC variants should express an anticoagulant activity, which is higher than the anticoagulant activity of the wild-type substance. Suitably, the present variants exhibit an anticoagulant activity which is enhanced at least about 400% or more, e.g. up to 1000%, or even up to 3000% over wild-type protein C.

Based on the above and similar principles, preferred mutations in the Gla-domain (SEQ ID NO:5) of variants of the present invention were determined. More specifically, in a theoretical paper by MacDonald et al (Biochemistry 1997; 36: 5120-5127) the sequences of all known Gla-domains were compared and an effort was made to correlate the sequences with the abilities of these Gla-domains to bind to negatively charged phospholipid. From this analysis, it was suggested that the great variation in affinities for negatively charged phospholipid among the various Gla domains was related to amino acid sequence differences mainly around residues at position 10 and 32 and 33.

In a previous paper by Shen et al (J Biol Chem 1998, 273: 31086-31091), several different mutants were created and tested following the theoretical considerations of MacDonald et al. The common theme for these mutants was to change position 11 from a serine (S) to a glycine (G) and position 32 from a glutamine (Q) to a glutamic acid (E, that will be converted to Gla in the mature protein) and position 33 from a asparagine (N) to an aspartic acid (D). In addition, positions 10 and 12 were changed one at the time, but not together. Thus, the mutants tested were E10G11E32D33 (EGED), Q10G11E32D33 (QGED), G11N12E32D33 (GNED) in addition to G11E32D33 (GED), E32D33 (ED) and E32 (E).

It was observed that QGED and GNED were essentially equally effective as anticoagulants and that both were more anticoagulant than wt APC. As compared to wt APC, both mutants bound phospholipid vesicles containing negatively charged phospholipid in a superior manner, and also bound Ca²⁺ more tightly. Even though the most efficient mutants of that study were more anticoagulant than wt APC, this was only found when low concentrations of phospholipid were used. Thus, it was suggested that, even though it was found that improved enzymatic activity of APC correlated with increased membrane affinity for all membranes used, the enhanced affinity of APC for negatively charged phospholipids only improved anticoagulant (enzymatic) activity of APC at low concentrations of negatively charged phospholipids.

Stimulated by the work of Shen et al, (J Biol Chem 1998, 273: 31086-31091) the present investigation was initiated. The idea was that possibly more efficient mutations could be created by combining mutations at positions 10, 11, and 12 into one variant and in addition to test if mutations at positions 23 and 44 could affect the efficiency of the mutant APC. Positions 32 and 33 were believed to be important from the work by Shen et al (J Biol Chem 1998, 273: 31086-31091) although it was never proven. The mutants tested by Shen et al, i.e. EGED, QGED, GNED in addition to GED, ED (positions 32, 33) and E (position 32) could not prove with certainty the importance of the positions 32 and 33 for the following reasons. The mutants EGED, QGED, GNED and GED were all more efficient than wt APC, but the two mutants ED and E were not more efficient. This raised the possibilities that the mutations around positions 10-12 were those that created the more efficient proteins and that the 32 and 33 mutations were not required. It was hypothesized, but not proven, that the mutations at positions 10-12 had to be combined with mutations at positions 32 and 33. However, it was clear from the Shen et al (J Biol Chem 1998, 273: 31086-31091) study that mutations at positions 32 and 33 alone were insufficient for the creation of protein C variants exhibiting enhanced anticoagulant activity. As will be demonstrated below, neither mutagenesis at positions 10-12 (the QGN variant) nor at positions 23, 32, 33, and 44 (the SEDY variant) did create molecules with more than slightly improved anticoagulant activity. Only the specific mutant (SEQ ID NO: 3) that contains all the above-identified modifications (designated QGNSEDY or “ALL”) was highly efficient.

As regards amino acid residues suitable for use to substitute wild-type residues at the above-identified positions of wild-type protein C, a comparison of amino acid sequences of different Gla-domains was performed, that included correlation analysis between these amino acid sequences and the phospholipid binding abilities of the different vitamin K-dependent proteins. This suggested that QGN was an interesting option for positions 10, 11, and 12, because both human protein S and bovine factor X comprise these sequences and both these proteins bind negatively charged phospholipid with high affinity. In many Gla domains, position 23 is occupied by a serine (S) residue and that is the reason why the wild-type residue of protein C was replaced with a serine residue when creating a suitable variant of the present invention. It is to be noted that modification of position 44 has not been considered before. However, since the only Gla domain that contains a histidine (H) residue at position 44 is the human protein C Gla domain, all other Gla domains having a tyrosine residue at position 44, it seemed logical that replacement of the histidine residue at position 44 with a tyrosine (Y) could be a useful modification.

Thus, a suitable strategy for selection of mutations in the Gla-domain is based on the fact that there are several vitamin K-dependent proteins having similar Gla-domains. In fact, all the Gla-domains have the same basic fold. The amino acid residues that are important for the folding of the domain are highly conserved, which includes a number of Gla-residues that bind calcium and thereby are crucial for the folding of the domain. Also some other amino acid residues are involved in the folding of the domain. Alignment of the sequences from all known Gla-domain-containing proteins demonstrate the natural variation of sequences of the Gla-domain and the conserved amino acid residues are highlighted in such an analysis. These amino acid residues tend to be located in the interior of the domain. In contrast, positions occupied by the exposed amino acid residues are more likely highly divergent and these positions are preferred positions for mutagenesis, as the mutations are less likely to cause folding problems. Amino acid residues at these positions are also highly variable in the family of Gla-domains. These positions are e.g. positions 10-12, 23, 32, and 33. Different Gla-domains have highly different affinities for negatively charged phospholipid membranes, which must be due to amino acid differences in the variable positions. By comparing the amino acid sequences of the Gla-domains with the affinities for negatively charged phospholipid, one can extract information that can be useful for the mutagenesis strategy, which is proven for the various protein C variants that have been prepared previously and have been discussed above. These include proteins mutated at positions 10-12, 23, 32, and 33. Many additional variants are possible where these positions are mutated to other amino acid residues than those already tried. In the selection of amino acids for replacement, one can try to stay within the family of amino acids but it might also be interesting to go beyond the family boundaries. The mutagenesis of position 44 from His to Tyr was done as all other Gla-domains have a Tyr at position 44.

The main object of modifying the Gla-domain is to obtain protein C variants with increased affinity for negatively charged phospholipid membranes. The advantage is that more APC will be present on the phospholipid membrane and thus the inhibitory effect on coagulation will be more pronounced. An advantage of this is that the effect of APC will be less dependent on the presence of cofactors, such as protein S and factor V. In many pathological situations, the cofactors are consumed by pathological proteolysis. The high efficiency of “super-APC” variants even in the absence of cofactors will be a distinct advantage over wt-APC.

From the above discussion it is evident that, even though the Gla-domain contains 45 amino acid residues, each of which could be modified independently or in combination, and the APC variant thereby produced would have to be characterized in a search for further variants having enhanced anticoagulant activity, such a search is indeed within reach for the skilled artisan. Moreover, based on the state of the art, e.g. using the variants specifically disclosed herein as precursors, further variants having essentially the same properties as the precursor variants (e.g. those variants specifically prepared in the experimental part), could be produced, e.g. by introducing one or a few conservative substitutions, or by introducing modifications in parts of the Gla-domain or other parts of the protein C molecule where such modifications would not affect the properties of the precursor that is intended to be modified. Such variants exhibiting essentially unchanged or the same properties as the present variants are considered to be equivalent to the present variants and thus to be encompassed by the present invention. The same is true for the SP-domain, at least if the mutagenesis targets are selected from amino acid residues nos. 290-320 or specifically amino acid residues nos. 300-314.

C. DNA Segments and Preparation Thereof

The present invention is also concerned with the deoxyribonucleic acid (DNA) segments or sequences related to the PC/APC variants, e.g. the structural genes coding for these variants, mutagenizing primers comprising the coding sequence for the modified amino acid stretch, etc.

In this connection, the well-known redundancy of the genetic code must be taken into account. That is, for most of the amino acids used to make proteins, more than one coding nucleotide triplet (codon) can code for or define a particular amino acid residue. Therefore, a number of different nucleotide sequences may code for a particular amino acid residue sequence. However, such nucleotide sequences are considered as functionally equivalent since they can result in the production of the same amino acid residue sequence. Moreover, occasionally, a methylation variant of a purine or pyrimidine may be incorporated into a given nucleotide sequence, but such methylations do not effect the coding relationship in any way. Thus, such functionally equivalent sequences, which may or may not comprise methylation variants, are also encompassed by the present invention.

A suitable DNA segment of the present invention comprises a DNA sequence, that encodes the modified (variant or mutant) PC/APC of the present invention, that is, the DNA segment comprises the structural gene encoding the modified PC/APC. However, a DNA segment of the present invention may consist of a relatively short sequence comprising nucleotide triplets coding for a few up to about 15 amino acid residues inclusive of the modified amino acid stretch, e.g. for use as mutagenizing primers.

A structural gene of the present invention is preferably free of introns, i.e. the gene consists of an uninterrupted sequence of codons, each codon coding for an amino acid residue present in the said modified PC/APC. However, the gene may also comprise introns and other control elements of gene expression occuring in the natural gene.

One suitable DNA segment of the present invention encodes an amino acid residue sequence that defines a PC/APC variant that corresponds in sequence to the wild-type human PC/APC except for at least one amino acid modification (insertion, deletion, substitution) in the amino acid sequence corresponding to the Gla-module of the wild-type protein and at least one amino acid modification (insertion, deletion, substitution) in the amino acid sequence corresponding to the SP-module of the wild-type protein.

Other suitable DNA segments encode PC/APC variants, wherein said modification(s) of the Gla-domain are contained in the amino acid residue sequence thereof at a position other than positions 10, 11, 28, 32, or 33. A preferred DNA-segment encodes a PC variant containing the modifications H10Q, S11G, S12N, D23S, Q32E, N33D, and H44Y or the modifications S11G, S12N, Q32E and N33D in its Gla-domain and modifications in an amino acid residue stretch of its SP-domain that comprises residues nos. 300-314, the modified stretch being comprised of WGYRDETKRNR (SEQ ID NO: 7).

In addition, the present invention is related to homologous and analogous DNA sequences that encode the present PC/APC variants, and to RNA sequences complementary thereto.

The present DNA segments can be used to produce the PC/APC variants, suitably in a conventional expression vector/host cell system, as will be explained further below (Section D).

As regards the DNA segments per se, these can be obtained in accordance with well-known technique. For instance, once the nucleotide sequence has been determined using conventional sequencing methods, such as the dideoxy chain termination sequencing method (Sanger et al., 1977), said segments can be chemically synthesized, suitably in accordance with automated synthesis methods, especially if large DNA segments are to be prepared. Large DNA segments can also be prepared by synthesis of several small oligonucleotides that constitute the present DNA segments followed by hybridization and ligation of the oligo-nucleotides to form the large DNA segments, well-known methods being used.

If chemical methods are used to synthesize the present DNA segments, it is of course easy to modify the DNA sequence coding for the wild-type PC/APC by replacement, insertion and/or deletion of the appropriate bases encoding one or more amino acid residues in the wild-type molecule.

Suitably, recombinant DNA technique is used to prepare the present DNA segments comprising a modified structural gene. Thus, starting with recombinant DNA molecules comprising a gene, i.e. cDNA encoding wild-type PC/APC, a DNA segment of the present invention comprising a structural gene encoding a modified PC/APC can be obtained by modification of the said recombinant DNA molecule to introduce desired amino acid residue changes, such as substitutions (replacements), deletions and/or insertions (additions), after expression of said modified recombinant DNA molecule. One convenient method for achieving these changes is by site-directed mutagenesis, e.g. performed with PCR-technology. PCR is an abbreviation for Polymerase Chain Reaction, and was first reported by Mullis and Faloona (1987).

Site-specific primer-directed mutagenesis is now standard in the art and is conducted using a synthetic oligonucleotide primer, which primer is complementary to a single-stranded phage DNA comprising the DNA to be mutagenized, except for limited mismatching representing the desired mutation(s). Briefly, the synthetic oligonucleotide is used as a primer to direct synthesis of a strand complementary to the phage DNA inclusive of the heterologous DNA and the resulting double-stranded DNA is transformed into a phage-supporting host bacterium. Cultures of the transformed bacteria are plated on top agar, permitting plaque formation from single cells that harbour the phage. In this method, the DNA which is mutated must be available in single-stranded form which can be obtained after cloning in M13 phages. Site-directed mutagenesis can also be accomplished by the “gapped duplex” method (Vandeyar et al., 1988; Raleigh and Wilson, 1986).

In accordance with a suitable embodiment of the present invention, site-directed mutagenesis is performed with standard PCR-technology (Mullis and Faloona, 1987). Examplary PCR based mutagenizing methods are described in the experimental part of the present specification. In these examples, the replication of the mutant DNA-segment is accomplished in vitro, no cells, neither prokaryotic nor eukaryotic, being used.

Obviously, site-directed mutagenesis can be used as a convenient tool for construction of the present DNA segments that encode PC/APC variants as described herein, by starting, e.g. with a vector containing the cDNA sequence or structural gene that encodes and expresses wild-type PC/APC, said vector at least being capable of DNA replication, and mutating selected nucleotides as described herein, to form one or more of the present DNA segments coding for a variant of this invention. Replication of said vector containing mutated DNA may be obtained after transformation of host cells, usually prokaryotic cells, with said vector. Illustrative methods of mutagenesis, replication, expression and screening are described in the experimental part of the present specification.

D. Preparation of PC/APC Variants

Such DNA segments, which comprise the complete cDNA sequence or structural gene encoding a PC/APC variant, can be used to produce the encoded variant by expression of the said cDNA in a suitable host cell, preferably a eukaryotic cell. Generally, such preparation of variants of the present invention comprises the steps of providing a DNA segment that encodes a variant of this invention; introduction of the provided DNA segment into an expression vector; introduction of the vector into a compatible host cell; culturing the host cell under conditions required for expression of the said variant; and harvesting the expressed variant from the host cell. For each of the above mentioned steps suitable methods are described in the experimental part of the present specification.

Vectors, which can be used in accordance with the present invention comprise DNA replication vectors, which vectors can be operatively linked to a DNA segment of the present invention so as to bring about replication of this DNA segment by virtue of the vector's capacity of autonomous replication, usually in a suitable host cell.

To achieve not only DNA replication but also production of the variant encoded by a DNA segment of the present invention, the said DNA segment is operatively linked to an expression vector, i.e. a vector capable of directing the expression of a DNA segment introduced therein. Replication and expression of DNA can be achieved from the same or different vectors.

The present invention is also directed to recombinant DNA molecules, which contain a DNA segment of the present invention operatively linked to a DNA replication and/or expression vector.

It is well known that the choice of a vector, to which a DNA segment of the present invention can be operatively linked, depends directly on the functional properties desired for the recombinant DNA molecule, e.g. as regards protein expression, and the host cell to be transformed. A variety of vectors commercially available and/or disclosed in prior art literature can be used in connection with the present DNA segments, provided that such vectors are capable of directing the replication of the said DNA segment. In case of a DNA segment containing a structural gene for a PC/APC variant, preferably, the vector is also capable of expressing the structural gene when the vector is operatively linked to said DNA segment or gene.

A suitable embodiment of the present invention is concerned with eukaryotic cell expression systems, suitably vertebrate, e.g. mammalian, cell expression systems. Expression vectors, which can be used in eukaryotic cells are well known in the art and are available from several commercial sources. Generally, such vectors contain convenient restriction sites for insertion of the desired DNA segment. Typical of such vectors are pSVL and pKSV-10 (Pharmacia, Sweden), pBPV1/pML2d (International Biotechnologies, Inc.), pXT1 available from Stratagene (La Jolla, Calif.), pJ5Eω available from The American Type Culture Collection (ATCC; Rockwille, Md.) as accession number ATCC 37722, pTDT1 (ATCC 31255) and the like eukaryotic expression vectors. In the experimental part of the present disclosure, pRc/CMV (available from Invitrogen, California, U.S.A.) has been used to obtain expression plasmids for use in adenovirus-transfected human kidney cells.

Suitable eukaryotic cell expression vectors used to construct the recombinant DNA molecules of the present invention include a selection marker that is effective in eukaryotic cells, preferably a drug resistance selection marker. A suitable drug resistance marker is the gene whose expression results in neomycin resistance, i.e. the neomycin phosphotransferase (neo) gene, Southern et al., J. Mol. Appl. Genet., 1:327-341 (1982). A further suitable drug resistance marker is a marker giving rise to resistance to Geneticin (G418). Alternatively, the selectable marker can be present on a separate plasmid, in which case the two vectors will be introduced by co-transfection of the host cell and selection is achieved by culturing in the appropriate drug for the selectable marker.

Eukaryotic cells, which can be used as host cells to be transformed with a recombinant DNA molecule of the present invention, are not limited in any way provided that a cell line is used, which is compatible with cell culture methods, methods for propagation of the expression vector and methods for expression of the contemplated gene product. Suitable host cells include yeast and animal cells. Vertebrate cells, and especially mammalian cells are preferred, e.g. monkey, murine, hamster or human cell lines. Suitable eukaryotic host cells include Chinese hamster ovary (CHO) cells available from the ATCC as CCL61, NIH Swiss mouse embryo cells NIH/3T3 available from the ATCC as CRL1658, baby hamster kidney cells (BHK) and the like eukaryotic tissue culture cell lines. In the experimental part of this specification, an adenovirus-transfected human kidney cell line 293 (available from American Type Culture Collection, Rockville, Md., U.S.A.) has been used.

To obtain an expression system in accordance with the present invention, a suitable host cell, such as a eukaryotic, preferably mammalian, host cell, is transformed with the present recombinant DNA molecule, known methods being used, e.g. such methods as disclosed in Graham et al., Virol., 52:456 (1973); Wigler et al., Proc. Nat'l. Acad. Sci. USA, 76:1373-76 (1979).

Thus, to express the DNA segment of the present invention in a eukaryotic host cell, generally, a recombinant DNA molecule of the present invention is used that contains functional sequences for controlling gene expression, such as an origin of replication, a promoter which is to be located upstream of the DNA segment of the present invention, a ribosome-binding site, a polyadenylation site and a transcription termination sequence. Such functional sequences to be used for expressing the DNA segment of the present invention in a eukaryotic cell my be obtained from a virus or viral substance, or may be inherently contained in the present DNA segment, e.g. when said segment comprises a complete structural gene.

A promoter that can be used in a eukaryotic expression system may, thus, be obtained from a virus, such as adenovirus 2, polyoma virus, simian virus 40 (SV40) and the like. Especially, the major late promoter of adenovirus 2 and the early promoter and late promoter of SV40 are preferred.

A suitable origin of replication may also be derived from a virus such as adenovirus, polyoma virus, SV40, vesicular stomatitis virus (VSV) and bovine papilloma virus (BPV). Alternatively, if a vector that can be integrated into a host chromosome is used as an expression vector, the origin of replication of the host chromosome may be utilized.

Even if eukaryotic expression systems are preferred, prokaryotic expression systems can also be used in connection with the present invention. Moreover, prokaryotic systems can advantageously be used to accomplish replication or amplification of the DNA-segment of the present invention, subsequently the DNA segments produced in said prokaryotic system being used for expression of the encoded product, e.g. in a eukaryotic expression system.

Thus, a prokaryotic vector of the present invention includes a prokaryotic replicon, i.e. a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, those embodiments that include a prokaryotic replicon also include a gene, whose expression confers drug resistance to a bacterial host transformed therewith. Typical bacterial drug resistance genes are those that confer resistance to ampicillin or tetracycline.

If a prokaryotic system is used, not only for DNA replication but also as an expression system, these vectors that include a prokaryotic replicon also include a prokaryotic promoter capable of directing the expression, i.e. transcription and translation, of the present DNA segment containing a structural gene, in a bacterial host cell, such as E. coli, transformed therewith. A promoter is an expression control element formed by a DNA sequence that permits binding of RNA polymerase and transcription to occur.

Promoter sequences compatible with bacterial hosts are typically provided in plasmid vectors containing convenient restriction sites for insertion of a DNA segment of the present invention. Typical of such vector plasmids are pUC8, pUC9, pUC 18, pBR322 and pBR329 available from BioRad Laboratories, Richmond, Calif., and pPL and pKK223 available from Pharmacia, Sweden.

Accordingly, to obtain a prokaryotic expression system, which can express the gene product of the present invention, appropriate prokaryotic host cells are transformed with a recombinant DNA molecule of the present invention in accordance with well known methods that typically depend on the type of vector used, e.g. as disclosed in Maniatis et al., Molecular Cloning, A Laboratory Manual, Cold Spring Harbor Laboratory, Cold Spring Harbor, N.Y. (1982).

It is of course necessary that successfully transformed prokaryotic or eukaryotic cells can be distinguished and separated from non-transformed cells. For this purpose, a variety of methods are known and have been described in prior art literature.

In accordance with one such method, the presence of recombinant DNA is assayed for by examining the DNA content of monoclonal colonies derived from cells which have been subjected to a transformation procedure. Such methods have been disclosed by Southern, J. Mol. Biol. 98:503 (1975) and Berent et al., Biotech., 3:208 (1985).

Successful transformation can also be confirmed by well-known immunological methods, e.g. using monoclonal or polyclonal antibodies specific for the expressed gene product, or by the detection of the biological activity of the expressed gene product.

Thus, cells successfully transformed with an expression vector can be identified by the antigenicity or biological activity that is displayed. For this purpose, samples of cells suspected of being transformed are harvested and assayed for either the said biological activity or antigenicity.

Such selected, successfully transformed cells are used to produce the desired PC/APC variants as disclosed above.

E. Assays for Biological Activity

Suitable methods for assaying the biological activity of the PC/APC variants of the present invention are based on plasma clotting systems, such as an APTT system, and on tests related to degradation of purified factor VIIIa and factor Va. Such methods are disclosed in more detail in the experimental part of the present specification.

F. Compositions

The present PC/APC variants are typically provided in a compositional form that is suitable for the intended use. Such compositions should preserve biological activity of the PC/APC variant and also afford stability thereof. Suitable compositions are therapeutic compositions that contain a therapeutically active amount of a variant according to the present invention, e.g. in combination with a physiologically tolerable carrier. Suitably, such compositions are lyophilized. In addition, said compositions could also contain a therapeutically active amount of a further active ingredient, such as protein S and/or Factor V, to enhance the anticoagulant activity thereof. Since protein C is a calcium dependent protein, suitably, the present compositions also contain divalent calcium, preferably in a physiological amount.

Since considerations to be taken into account in connection with design of compositional forms in general, and specifically therapeutic compositions, are well known to the skilled artisan, there is no need to describe these in more detail.

G. Therapeutic Methods

According to the present invention, it has been shown that the present PC/APC variants exhibit an enhanced anticoagulant activity. Thus, the present invention is also concerned with methods for inhibiting coagulation in an individual, e.g. a human, said methods comprising administering to said individual a composition comprising a therapeutically effective amount of a variant PC/APC of the present invention. Conditions that could be treated are disclosed elsewhere in this specification.

As for compositions, considerations to be taken into account in connection with design of therapeutic methods, e.g. suitable dosage ranges and administration routes, are well known to the skilled artisan, and, thus, there is no need to describe these methods in more detail.

Briefly, however, the present protein C variants can be administered via different routes of administration. For instance, the protein C variants may be prepared as compositions for parenteral administration, for oral administration, or for nasal administration. Thus, to ensure efficient delivery into the blood stream, the protein C variants could be administered by intravenous injection, continuous infusion, bolus injection or combinations thereof. Alternatively, the protein C variants could be administered subcutaneously if slower release into the blood stream is desired.

An appropriate dose of the protein C variant can easily be determined by the attending physician taking into account various circumstances, such as age, sex and overall health status of the individual to be treated. An effective dose should give rise to plasma ranges of 0.02 ng/ml to less than 100 ng/ml, suitably 0.2-50 ng/ml, preferably 2-60 ng/ml and specifically 40-50 ng/ml. Thus, injection of a dose of 0.01 mg/kg/day to at least about 1.0 mg/kg/day, one to six times a day for one to ten days could be used in many methods for treatment of thromboembolic conditions in order to inhibit undesired blood coagulation.

Preferably, preparations for parenteral administration are comprised of liquid solutions or suspensions in aqueous physiological buffer solutions. For oral administration, tablets or capsules are suitable unit dosage forms.

H. Discussion

The present invention is related to PC-variants that contain at least one modification in each of the Gla- and SP-domains of wild-type PC.

A specific variant according to the present invention that has been prepared in the experimental part of this specification combines the mutations in the Gla-domain of a variant containing the mutated sequence of SEQ ID NO: 5 with mutations in the SP-domain in the amino acid residue stretch comprising the positions 300-314, the mutated sequence corresponding to the mutated sequence of SEQ ID NO: 7. A further variant contains the afore-said mutations in the SP-domain, i.e. the mutated sequence of SEQ ID NO:7, in combination with the mutations S11G, S12N, Q32E, and N33D in the Gla-domain.

Previously, the Inventor has studied PC-variants containing mutations only in the Gla-domain and also PC-variants containing mutations only in the SP-domain. The latter PC-variants (“SP-mutants”) have been disclosed in WO 98/44 000, whereas the “Gla-mutants” are disclosed in U.S. provisional patent application No. 60/272,466 filed on Mar. 2, 2001.

H(1) Gla-Mutations

In the above-mentioned studies concerned with Gla-mutants, it was found that a variant containing the mutated Gla-domain having the sequence of SEQ ID NO: 5 and designated QGNSEDY (ALL) is more anticoagulant than wt APC and is also more anticoagulant than previously reported Gla-domain mutants such as GNED or QGED (described by Shen et al, supra). It is quite a surprise that this variant exhibits a much enhanced activity, i. a. since neither of the two variants QGN and SEDY exhibits any, or only exhibits a slightly, increased anticoagulant activity or increased affinity for negatively charged phospholipid membranes. This suggests that the membrane-binding ability of the Gla-domain is very complex and not easily affected by single amino acid replacements. Only when multiple areas of the Gla-domain are mutated, it is possible to obtain a unique variant like QGNSEDY (ALL) that exhibits much enhanced phospholipid affinity and much increased anticoagulant activity.

The anticoagulant activity of QGNSEDY (ALL) is potentiated by protein S, which stands in contrast to the activity of a chimeric APC variant described by Smirnov and Esmon in U.S. Pat. No. 5,837,843. This variant is a hybrid between protein C and prothrombin, wherein the prothrombin Gla-domain is replacing the corresponding Gla-domain in protein C (PC). Although, due to enhanced phospholipid binding, this PC/APC variant is more anticoagulant than wild-type APC, its activity is not potentiated by protein S.

Also EP 0 296 413 A2 is concerned with protein C hybrids, not only between prothrombin and PC but also between FVII, FIX, or FX and PC. These variants contain the Gla-domain from prothrombin, FVII, FIX, or FX and the rest from PC. However, in these variants the Gla-domain has been limited to the first N-terminal 43 amino acid residues and thus, these variants do not contain a modified amino acid residue at position 44 of wt protein C. Although it is stated therein, that these variants have improved activity against blood clot formation or improved fibrinolysis accelerating effect, these variants have not been well characterized as regards such activities. Only a FX/PC hybrid has been prepared and characterized and this hybrid was not found to have improved anticoagulant properties over wt PC apart from improved inactivation of factor Va.

A further quite unexpected advantage with the present variant QGNSEDY (ALL) is that since it is able to cleave FVa at Arg306, it is indeed able to cleave a mutated FVa (designated. FV:Q506 or FV Leiden) that is mutated at the main cleavage site attacked by APC, i.e. at position Arg506. This mutated factor Va is present in the common blood coagulation disorder designated APC-resistance. Accordingly, the ability of QGNSEDY (ALL) to cleave FVa at Arg306 is an advantage over wild-type APC that is very poor in cleaving the Arg306, which is the site that when cleaved results in complete inactivation of FVa. Thus, contrary to wild-type APC, the present variant QGNSEDY (ALL) is capable of cleaving and inactivating activated FV:Q506. In contrast to the cleavage at Arg506, the cleavage at Arg306 is potentiated by protein S. However, a further advantage of the present variant QGNSEDY (ALL) is that it cleaves activated FV:Q506 even in absence of protein S. Yet, this cleavage is stimulated by protein S, even though protein S is not required. The ability of the present variant QGNSEDY (ALL) to cleave activated factor V at Arg306, makes it attractive as an anticoagulant also for patients with APC-resistance.

H(2) SP-Mutants

In WO 98/44 000, the Inventor reports research concerned with modifications in the SP-domain of PC, and specifically with a modified SP-domain containing the mutated sequence of SEQ ID NO:7.

This is a shortened amino acid sequence (SEQ ID NO: 7) in comparison to wild-type human protein C that is identical with the corresponding amino acid sequence of the bovine SP module. Since a comparison between the human, bovine, rat, and mouse sequences of the SP module revealed that the rat and mouse PC/APC molecules were more similar to human PC/APC than was the case with bovine PC/APC, mutants were prepared and investigated, which mutants comprised deletion and substitution mutations in human PC/APC making the 300-314 amino acid sequence identical with the corresponding sequence of bovine PC/APC. Vice versa, insertion and substitution mutations were introduced into bovine PC/APC to extend the bovine sequence corresponding to amino acid numbers 300-314 of human PC/APC and to make that sequence identical with the human amino acid sequence No. 300-314. In the experimental part of the present specification and of WO 98/44000, isolation and characterization of mutants of human PC/APC and bovine PC/APC are described. Using standard PCR technology (Mullis and Faloona (1987), Meth. Enzymol. 155, 335-350), the above deletion, substitution and insertion mutations were made in the cDNA's of human PC/APC and bovine PC/APC. Thus, after expression of these mutated cDNA's in a eukaryotic system, a mutated human PC/APC molecule comprising the sequence of SEQ ID NO:7 and a mutated bovine PC/APC molecule comprising the sequence of SEQ ID NO:6 were produced and purified to homogeneity. In addition, the cDNA's of wild-type human and bovine protein C/APC were expressed in this eukaryotic system and the expression products were purified to homogeneity. To characterize the purified wild-type PC/APC molecules and variants thereof obtained in these procedures, these molecules were activated by thrombin and the thrombin activation products were separated by S-Sepharose chromatography. The functional properties of the isolated PC/APC molecules were then characterized. The different PC/APC contructs, obtained by expression of the above mentioned cDNA's and a subsequent purification procedure, are referred to as follows: wt-hPC/APC, the wild-type human PC/APC; Δ-hPC/APC, human protein C comprising the shortened sequence corresponding to sequence of SEQ ID NO: 7; wt-bPC/APC, wild-type bovine PC/APC; ins-bPC/APC, bovine PC/APC comprising an extended sequence corresponding to sequence of SEQ ID NO: 6. These mutants, Δ-hPC/APC and ins-bPC/APC, are also designated human APC-SP and bovine APC-SP, resp., the latter designations being used mainly in the following Example I and the Figures referred to in this example.

As is obvious from Example 1, below, on standard SDS-polyacrylamide gel electrophoresis, these recombinant PC/APC constructs had the expected molecular weights when run under both reducing and non-reducing conditions. The amidolytic activity, i.e. the proteolytic activity against a low molecular weights substrate, such as S-2238 (Chromogenix AB, Mölndal, Sweden) was characterized and it was observed that the mutated human PC/APC (Δ-hPC/APC) had much higher activity against the substrate than wild-type human PC/APC. The bovine mutation (ins-bPC/APC) on the other hand, had much lower activity against the synthetic substrate, which suggested that the deletion/insertion mutations affected the catalytic site of the PC/APC, even though the mutations were positioned at some distance from the active site.

Thus, this previous research unexpectedly revealed that mutations in the SP-module of PC/APC, which mutations are positioned at some distance from the active site of PC/APC could give rise to PC/APC variants having enhanced anticoagulant activity due to enhanced proteolytic, and more specifically enhanced amidolytic, activity. The conclusion that the present mutations are not located within or adjacent to this active site is based on a published hypothetical molecule model of APC and the elucidated model of the three-dimensional structure of the SP-module of APC, which is disclosed in EMBO Journal, 1996, 15: 6810-6821 (Mather et al.). From these models, it appears that the mutations contained in the above-mentioned constructs are located in loop 5 of the SP-module, which loop is not directly in contact with the active site region.

In the experimental part of the present specification, the kinetics of the synthetic substrate cleavages of the above recombinant PC/APC molecules have been characterized as reported in WO 98/44000, i.e. the values of Km, Vmax and kcat were determined by changing the substrate concentration, as elucidated in more detail in Example 1, below. As reported in WO 98/44000, it was found that the value of Km was decreased, suggesting that the affinities of the various APC-molecules for the substrate were higher. Moreover, the values of Vmax were very different, Δ-hPC/APC having at least a 7-fold higher value of Vmax than the wt-hPC/APC; ins-bPC/APC on the other hand, had a distinctly lower value of Vmax whereas the value of Km was virtually unaffected. These results suggest that the increased activity of Δ-hPC/APC was due to a combination of increased catalytic activity and increased affinity for the substrate caused by the mutations.

In addition, as reported in WO 98/44000 and in Example 1, below, the anticoagulant activities of the above mentioned mutated PC/APC molecules have been measured in a plasma clotting system based on the APTT (activated partial thromboplastin time) reaction (activation by intrinsic pathway). It was observed in these tests, that, when added to human plasma, the Δ-hPC/APC had enhanced anticoagulant response as compared to wt-hPC/APC. In the absence of added bovine protein S, both wt-bPC/APC and ins-bPC/APC had very poor anticoagulant response, whereas both these bovine recombinant compounds expressed distinct anticoagulant activity when bovine protein S was also included in the reaction mixture.

The above results indicate that the reported deletion-mutation in the human APC led to enhanced activity against the natural substrates present in human plasma (FVa and FVIIIa) whereas the reported insertion-mutation in bovine APC did not significantly affect the reactivity against the natural substrates, even though the activity against the synthetic substrates was impaired. To confirm that the deletion mutation in human APC indeed led to increased proteolytic activity against the natural substrate FVIIIa, the effect of the recombinant APCs in a FVIIIa degradation system using purified components (previously described system, Shen and Dahlbäck, J. Biol. Chem. 1994, 269:18735-18738) was investigated as reported in WO 98/44000. The system included FIXa, FVIIIa, phospholipid vesicles and calcium, and the activity of FVIIIa was measured by the addition of FX and, after a short incubation time, also addition of a synthetic substrate against FXa. The effect of the various APC molecules was tested by the addition of APC together with its synergistic cofactors protein S (of the same species as the APC) and bovine FV. In this system it was obvious that the Δ-hPC/APC had higher activity than wt-hPC/APC, whereas the two bovine PC/APCs were relatively similar to each other. As regards the degradation of purified FVa, the various APCs were not tested but it is expected that Δ-hPC/APC will have higher activity than wt-hPC/APC. Since the changes introduced in to the human and bovine APCs might influence the rate of inhibition, the rate of inhibition of the mutated APC molecules was tested in human plasma. Thus, APC was added to plasma and at various intervals, the remaining amidolytic activity was measured. It was found that the mutated human molecule had the same half-life as the wild-type human APC suggesting that the mutation did not affect the rate of inhibition by serpins. To test this further, the rate of inhibition of mutated and wild-type APC by purified PCI and α1AT was tested and found to be essentially identical. Bovine APC and the mutated bovine APC on the other hand were not inhibited by α1AT, which demonstrates that the hypothesis about the mutated region being involved in determining the rate of inhibition was not correct i.e. that the explanation for the different inhibition pattern of human and bovine APC was not caused by the identified sequence difference but by another sequence difference yet to be defined.

In conclusion, the results reported in Example I demonstrate that the deletion-mutation in hAPC led to a molecule which had higher catalytic activity against the natural substrates FVIIIa and FVa as well as against low molecular substrates, whereas the mutation did not affect the rate of inhibition by serpins.

H(3) Combined Gla- and SP-Domain Mutants

Based on his findings for the Gla- and SP-modules the Inventor realized that a PC/APC variant that contains modifications both in the Gla-domain and in the SP-domain could provide PC/APC variants having improved properties, not only over wt PC/APC but preferably also over the above-mentioned Gla- and SP-mutants of PC/APC.

Provided that the combination of a modified Gla-domain and a modified SP-domain would not lead to interactions that could abolish any improved properties conferred on said mutants by the modifications in the Gla-domain or in the SP-domain, said combination mutants would exhibit further enhanced anticoagulant activity and/or a combination of properties that are improved over wt PC/APC, which combination is not exhibited by anyone of the Gla- and SP-mutants.

For instance, as indicated in H(1) above, an APC variant having enhanced ability to cleave FV Leiden and also enhanced anticoagulant activity due to enhanced proteolytic, e.g. amidolytic, activity could be obtained.

Accordingly, as illustrated in Example 8, the Inventor has prepared a preferred PC/APC variant that contains the modified Gla-domain of SEQ ID NO:5 and an SP-domain that contains the modified sequence of SEQ ID NO:7. As shown in Example 8, in an APTT-test this variant has much enhanced anticoagulent activity over wt PC/APC.

In addition, the Inventor has prepared a PC/APC variant wherein the Gla-domain contains the mutations G11N12E32D33 and the SP-domain contains the modified sequence of SEQ ID NO:7. This variant also exhibits improved anticoagulant activites over wt PC/APC (cf. Example 9).

I Potential Use of the Present PC/APC Variants

It is obvious that a recombinant protein C molecule which after its activation to APC exhibits enhanced anticoagulant activity has great potential use both as a possible therapeutic compound and as a reagent to be used in various biological assays for other components of the protein C system. In accordance with the present invention it has been shown that if mutations are present both in the Gla module and the SP-module of the protein C molecule, a variant protein C can be obtained that has substantially enhanced anticoagulant activity, e.g. due to enhanced membrane-binding activity and to enhanced proteolytic, e.g. amidolytic, activity as well. Thus, it can be expected that a systematic search for such mutations could produce other protein C molecules with even better properties. For instance, by selection of specific mutations in the Gla-domain, it could become possible to design APC molecules with highly specific functions, e.g. additional molecules that cleave FVa at Arg306, and thus to produce additional APC variants which also work well to degrade said mutated FV which is present in the blood coagulation disorder APC-resistance. Selection of specific mutations in the SP-domain could make it possible to design APC molecules that mainly work against FVIIIa or mainly cleave FVa.

It is envisioned that the present protein C variants expressing enhanced anticoagulant activity will be useful in all situations where undesired blood coagulation is to be inhibited. Thus, the present variants could be used for prevention or treatment of thrombosis and other thromboembolic conditions. Illustrative of such conditions are disseminated intravascular coagulation (DIC), arterioschlerosis, myocardial infarction, various hypercoagulable states and thromboembolism and also sepsis and septicaemia. The present variants could also be used for thrombosis prophylaxis, e.g. after thrombolytic therapy in connection with myocardial infarction and in connection with surgery and for treatment of APC-resistance (inherited or acquired) or protein C deficiency (inherited or acquired). A combination of the present protein C variants and protein S (wild-type protein S or a variant thereof) could be useful, which combination also could include Factor V exhibiting activity as a cofactor to APC.

Moreover, because APC has multiple activities, e.g. it manifests not only antithrombotic activity but also profibrinolytic, anti-inflammatory, and antiapoptotic activities, the present APC variants have a potential role in the treatment of various complex medical disorders, including severe sepsis, thrombosis and stroke. APC is a systemic anticoagulant and also an antiflammatory factor and in animal models of sepsis, ischemic injury and stroke, it has been found to reduce organ damage. It also substantially reduces mortality in patients with severe sepsis.

A further potential use of the APC variants is in the treatment of subjects having a neuropathological disorder or brain inflammatory disease, e.g. neurodegenerative diseases with different types of neuronal dysfunction, such as stroke, Alzheimer's disease and various antoimmune diseases.

It is envisioned that the present PC/APC variants could be used in treatment of the same conditions as native APC.

As regards diagnostic use of the present PC/APC variants, there is a great need for improved functional assays for protein S and also for the anticoagulant activity of factor V. It is likely that a mutated APC with enhanced anticoagulant activity due to enhanced membrane-binding and enhanced proteolytic, suitably amidolytic, activity will be very useful in such assays because such APCs will give stronger signals and this will lead to increased signal to noise ratios in different assays. For the SP-mutants, this is confirmed by the initial in vitro characterization of the mutated APC molecules reported in Example 1 that shows that the amidolytic activity is much higher for the mutant hAPC disclosed therein than for normal APC and also that the anticoabulant effect is higher for said mutant hAPC than for normal APC. The interaction of this mutated molecule with its cofactors protein S and intact FV appeared unaffected by the mutations in the SP-module which suggests that the concept of using the mutated hAPC (Δ-hAPC) in in vitro tests is correct.

It could also be possible to combine the combination of mutations in the Gla-module and the SP-module with additional mutations in other parts of protein C to produce protein C with very unique properties. Scientists at Ely Lilly (Ehrlich et al, Embo. J. 1990, 9:2367-2373; Richardson et al, Nature 1992, 360:261-264) and also other groups have already shown that mutations around the activation peptide region yielded protein C which was easily activated even in the absence of TM (thrombomodulin). Similarly, another set of mutations in the activation peptide region led to a protein C. molecule which was secreted in active form from the synthesizing cells (Ehrlich et al, J. Biol, Chem. 1989, 264:14298-14304). Also combinations of the present mutations with future mutations that may enhance the interactions between APC and its cofactors, are envisioned.

The present invention is of course directed to protein C variants defined herein irrespective of the mode of production thereof. In the previous sections, e.g. in section D, some suitable methods are disclosed.

However, other methods such as methods concerned with transgenic animals are foreseen to be useful. For instance, it is referred to Velander, et al., “Transgenic Livestock as Drug Factories” in Scientific American, January 1997, wherein a transgenic pig producing human protein C in her milk is disclosed. Thus, it seems likely that transgenic animals producing the present protein C variants could be obtained.

### EXPERIMENTAL PART

In the following examples suitable embodiments are disclosed that illustrate the present invention. However, these examples should not be construed as limiting the invention. Unless otherwise stated therein, human PC/APC variants have been prepared and human coagulation factors, plasma, etc. have been used.

In these examples, the following materials were used.

Human α1-antitrypsin (α1AT) and Protein C inhibitor (PCI) were kind gifts from Drs. Carl-B. Laurell and Margareta Kjellberg, respectively (Dept. of Clinical Chemistry, University Hospital, Malmö, Sweden). HPC₄ immunoaffinity columns were obtained from Dr. Charles T. Esmon (Howard Hughes Medical Institute, Oklahoma Medical Research Foundation, USA). Fast Flow Q-Sepharose (FFQ) and Octonative M (as source of factor VIII) were purchased from Pharmacia, Sweden. Lipofectin and Geneticin (G418) are available from Life Technologies AB, Sweden, and Dulbecco's Eagle's modified medium (DMEM) is available from Gibco Corp. Purified bovine factor IXa, factor X, phospholipid vesicles and the chromogenic substrate S-2222 were generous gifts from Dr. Steffen Rosén at Chromogenix AB, Sweden. Hirudin was obtained from Sigma Chemical Co., USA, and D-Phe-Pro-Arg Chloromethyl Ketone (PPACK) from Calbiochem, USA. Bovine factor V, α-thrombin, and human protein S as well as bovine protein S were purified according to previously described methods (Dahlbäick, et al., 1990; Dahlbäck and Hildebrand, 1994).

### Example 1

### SP-Mutants of Protein C

This example corresponds to Example 1 of WO 98/4400.

(a) Site Directed Mutagenesis

A full-length human protein C cDNA clone, which was a generous gift from Dr. Johan Stenflo (Dept. of Clinical Chemistry, University Hospital, Malmö, Sweden), and a full-length bovine protein C cDNA clone, kindly provided by Dr. Donald Foster (ZymoGenetics, Inc., USA) were separately digested with the restriction enzymes HindIII and XbaI and the resultant restriction fragment comprising the complete PC coding region, either human or bovine, that is full length protein C cDNA, was cloned into a HindIII- and XbaI-digested expression vector pRc/CMV.

The resultant expression vectors containing the coding sequences for wild-type human or bovine protein C were used for site-directed mutagenesis of the SP-module of protein C, wherein a PCR procedure for amplification of target DNA was performed as described below and as shown in the following reaction scheme (Scheme I). The nucleotide sequences of the primers used in this procedure are listed in Table I below.

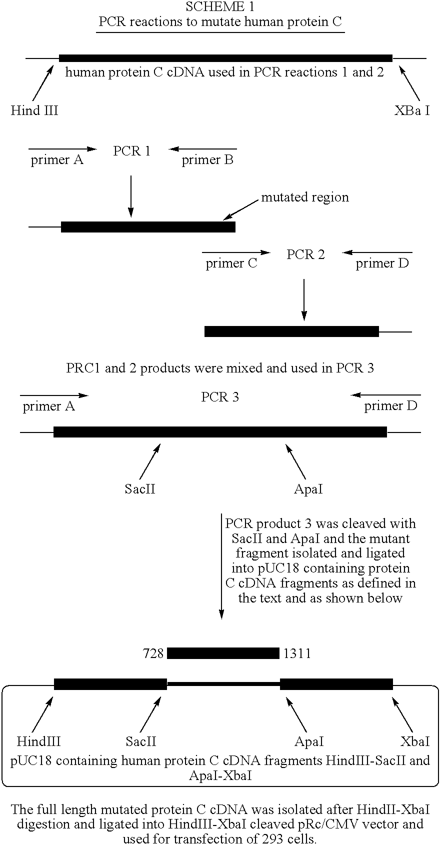

To obtain a mutagenized human protein C cDNA, a fragment of human Protein C cDNA containing the coding region from the 5′ terminal amino acid up to position 313 was amplified with the use of intact human protein C cDNA as a template and a pair of primers A and B. primer B being the mutagenic oligonucleotide (PCR1 of Scheme 1). A second fragment of human Protein C cDNA containing remaining amino acids after position 303 and, thus, partly overlapping the first fragment, was amplified with the use of intact human protein C cDNA as a template and a pair of primers C and D, primer C being the mutagenic oligonucleotide (PCR2 of Scheme I).

From the above PCR amplification procedures, two partly overlapping, doublestranded cDNA fragments were obtained, which both contain the mutagenized DNA sequence. These two cDNA fragments were used as templates together with two primers A and D in a further PCR procedure to amplify a full length human protein C cDNA containing the desired mutated amino acids (PCR3 of Scheme I).

The reagent mixture for each of the above PCR reactions was 100 μl containing 0.25 μg of template DNA, 200 μM each of the deoxyribonucleoside triphosphates (dNTP: dATP/dCTP/dGTP/dTTP), 0.5 μM of each primer and 2.5 U of Pwo-DNA polymerase (Boehringer Mannheim) in Tris-HCl buffer (10 mM Tris, 25 mM KCl, 5 mM (NH₄)₂SO₄, and 2 mM MgSO₄, pH 8.85). The sample was subjected to 30 cycles of PCR comprised of a 2 min denaturation period at 94° C., a 2 min annealing period at 55° C. and a 2 min elongation period at 72° C. After amplification, the DNA was subjected to electrophoresis on 0.8% agarose gel in 40 mM Tris-acetate buffer containing 1 mM EDTA. All PCR amplification products were purified by using JET Plasmid Miniprep-Kit (Saveen Biotech AB, Sweden).

The resultant human protein C cDNA containing the desired mutations was digested with SacII and ApaI, and then the fragment from the SacII and ApaI digestion (nucleotides 728-1311) was cloned into the vector pUC18 which contains intact human protein C fragments (HindIII-SacII, 5′ end-nucleotide 728; and ApaI-XbaI, nucleotide 1311-3′ end) to produce human protein C full length cDNA comprising the desired mutations, viz. coding for a human protein C mutant comprising the mutated sequence of SEQ ID NO:7 instead of the human wild-type sequence of SEQ ID NO:6.

In addition, bovine protein C cDNA was mutagenized and the mutated cDNA was amplified essentially as disclosed above, except that different primers and templates were used. The PCR amplification product of bovine protein C cDNA containing the desired mutations was cleaved with SalI and BglII, and the fragment from digestion with SalI and BglII (nucleotides 600-1123) was cloned into a vector pUC 18 containing intact bovine protein C fragments (HindIII-SalI, 5′ end-nucleotide 600 bp; and BglII-XbaI, nucleotide 1123-3′ end) to produce mutated bovine protein C full length cDNA in the vector pUC 18, whereafter HindIII and XbaI were used to cleave bovine protein C full length cDNA containing the desired mutations, viz. coding for a bovine protein C mutant comprising the mutated sequence of SEQ ID NO:6 instead of the bovine wild-type sequence of SEQ ID NO:7.

Then, each of the above mutated human and bovine protein C cDNA's was digested with HindIII and XbaI and the appropriate restriction fragment was cloned into the vector pRc/CMV, which had been digested with the same restriction enzymes. The vectors obtained were used for expression of mutated human or bovine protein C in eukaryotic cells.

Before transfection of the appropriate host cells, all mutations were confirmed by DNA sequencing by the dideoxy chain termination method of Sanger et al., supra.

For the above site-directed mutagenesis procedure, the following oligonucleotide primers listed in Table I in the 5′ to 3′ direction were used.

TABLE I

| | |
|---|---|
| Primer | |
| designa- | |
| tion | Nucleotide sequence |

| A | 5′-AAA TTA ATA CGA CTC ACT | (SEQ ID NO: 34) |
|---|---|---|
| | ATA GGG AGA CCC AAG CTT-3′ | |
| B | 5′-GTT TCT CTT GGT CTC GTC | (SEQ ID NO: 35) |
| | ACG GTA GCC CCA GCC CGT | |
| | CAC GAG-3′ | |
| C | 5′-CGT GAC GAG ACC AAG AGA | (SEQ ID NO: 36) |
| | AAC CGC ACC TTC GTC CTC-3′ | |
| D | 5′-GCA TTT AGG TGA CAC TAT | (SEQ ID NO: 37) |
| | AGA ATA GGG CCC TCT AGA-3′ | |
| E | 5′-GGC CTC CTT CTC TCG GCT | (SEQ ID NO: 38) |
| | GCT GTG GTA GCC CCA GCC | |
| | CGT CAC-3′ | |
| F | 5′-CAC AGC AGC CGA GAG AAG | (SEQ ID NO: 39) |
| | GAG GCC AAG AGA AAC CGC | |
| | ACC TTC-3′ | |
| Primers A-D were used to mutagenize and amplify human protein C cDNA, as disclosed above. To mutagenize and amplify bovine protein C cDNA, likewise, two pair of primers were used, viz, primers A and E and primers F and D, primers E and F being mutagenic primers. The nucleotide sequences of these primers are related to parts of the vector nucleotide sequence or parts of the protein C cDNA nucleotide sequence as explained below. | | |
| Primer A corresponds to nucleotides 860-895 in the vector pRc/CMV and provides a HindIII restriction site between the pRc/CMV vector DNA and the protein C cDNA. | | |
| Primer B corresponds to a partial, modified antisense nucleotide sequence of human protein C cDNA, the modified sense sequence coding for: LVTGWGYRDETKRN (SEQ ID NO: 40). | | |
| This amino acid residue sequence corresponds to a modified sequence of human protein C from amino acid residue number 296 to 313, inclusive, wherein the sequence of residues 303-310 contains mutations. i.e. the residues 303, 304, 305 and 308 are deleted and residues 307 and 310 are substituted, the resulting sequence RDET (SEQ ID NO: 43) being identical with the corresponding part of bovine protein C (residue numbers 305-308). | | |
| Primer C corresponds to a partial modified nucleotide sequence of human protein C cDNA coding for: RDETKRNRTFVL (SEQ ID NO: 41). | | |
| This amino acid residue sequence corresponds to a modified sequence of human protein C from amino acid residue number 303 to 318, inclusive, which contains the same mutations as disclosed for primer B above, i.e. the residue numbers 303-305 and 308 are deleted and residue numbers 307 and 310 are substituted. Thus, primer C encodes a shortened sequence RDET which is identical with the corresponding sequence of bovine protein C. | | |
| Primer D corresponds to the antisense sequence to the sequence of nucleotides 984-1019 in the vector pRc/CMV and provides a XbaI restriction site between the pRc/CMV vector DNA and the protein C cDNA. | | |
| Primer E corresponds to a partial modified antisense nucleotide sequence of bovine protein C cDNA, the modified sense sequence coding for: VTGWGYHSSREKEA (SEQ ID NO: 42). | | |
| This amino acid residue sequence corresponds to a modified sequence of bovine protein C from amino acid residue number 299 to 308, inclusive, wherein the sequence corresponding to residue numbers 305-308 (RDET) (SEQ ID NO:43) contains mutations, viz. four insertions and two substitutions, the mutated sequence being HSSREKEA (SEQ ID NO: 44) which is identical with the corresponding part of human protein C (residues numbers 303-310). | | |
| Primer F corresponds to a partial modified antisense nucleotide sequence of bovine protein C cDNA coding for: HSSREKEAKRNRTF (SEQ ID NO: 45). This amino acid residue sequence corresponds to a modified sequence of bovine protein C from amino acid residue number 305 to 314, inclusive, which contains the same mutations between positions 305 and 308 as stated for primer E above. Thus, primer F encodes an extended sequence HSSREKEA (SEQ ID NO: 46) which is identical with the corresponding | | |
| # sequence of human protein C. | | |

(b) Production of Stable Transformants Producing Variant or Wild-Type Protein C.

To produce stable transformants producing variant or wild-type protein C, adenovirus-transfected human kidney cell line 293, was grown in DMEM medium containing 10% of fetal calf serum, 2 mM L-glutamine, 100 U/ml penicillin, 100 U/ml streptomycin and 10 μg/ml vitamin K₁, and transfected with an expression vector comprising wild-type or mutagenized protein C cDNA from step (a). The transfection was performed in accordance with the Lipofectin method as described earlier (Felgner et al., 1987). In brief, 2 μg of vector DNA, which was diluted to 100 μl with DMEM containing 2 mM of L-glutamine, was mixed with 10 μl Lipofectin (1 μg/μl) that was diluted to 100 μl with the same buffer. The mixture was kept at room temperature for 10-15 min and was diluted to 1.8 ml with the medium, and then added to the cells (25-50% confluence in a 5-cm Petri dish) that had been washed twice with the same medium.

(c) Expression of variant or wild-type protein C. The transfected cells from section (b) were incubated for 16 hours, whereafter the medium was replaced with complete medium containing 10% calf serum and the cells were incubated for additional 48-72 hrs. The cells were then trypsinized and seeded into 10-cm dishes contaning selection medium (DMEM comprising 10% serum, 400 μg/ml G418,2 mM L-glutamine, 100 U/ml penicillin, 100 U/ml streptomycin and 10 μg/ml vitamin K₁) (Grinnell, et al. 1990). G418-resistant colonies were obtained after 3-5 weeks selection. From each DNA transfection procedure, 24 colonies were selected and grown until confluence. All colonies were screened by dot-blot assays using monoclonal antibody HPC₄ (for human protein C) or monoclonal antibody BPC₅ (for bovine protein C) to examine the protein C expression. High expression cell colonies were selected and grown until confluence in the selection medium. Thereafter, these cells were grown in a condition medium (selection medium lacking serum) to iniate expression of protein C or a variant thereof, which medium, like the selection medium, was replaced every 72 h. After a suitable time period, the condition medium containing the respective expression product was collected for purification of said product in section (d) below.

(d) Purification of Recombinant Wild-Type and Mutated Proteins

(i) Bovine recombinant protein C and its mutant were purified by the method described previously (Yen et al., 1990). Five mM of EDTA and 0.2 μM of PPACK were added to the condition medium collected in section (c). The medium was then applied to a Pharmacia FFQ anion-exchange column and eluted with a CaCl₂ gradient (starting solution, 20 mM Tris-HCl/150 mM NaCl, pH 7.4; limiting solution, 20 mM Tris-HCl/150 mM NaCl/30 mM CaCl₂, pH 7.4) at room temperature. The CaCl₂ was removed by overnight dialysis (20 mM Tris-HCl, 150 mM NaCl, pH 7.4) in combination with Chelex 100 treatment. The dialysate was then applied to a second FFQ column to readsorb protein C or its mutant to the column, whereafter protein was eluted with a NaCl gradient solution (starting solution 20 mM Tris-HCl/150 mM NaCl, pH 7.4; limiting solution, 20 mM Tris-HCl/500 mM NaCl, pH 7.4).

(ii) Culture medium obtained in section (c) from transformants producing human wild-type or mutant protein C was first subjected to column purification and, then, applied to an affinity column carrying monoclonal antibodies HPC₄ as described earlier (Rezaie and Esmon, 1994) except for slight modifications (He et al., 1994).

The purified proteins obtained in (i) and (ii) were concentrated on YM 10 filters (Amicon), dialyzed against TBS buffer (50 mM Tris-HCl and 150 mM NaCl, pH 7.4) for 12 hrs and stored at −80° C. until use thereof.

The purity and homogeneity of the above wild-type and mutant protein C's were established by SDS-PAGE. This electrophoresis procedure was run as a polyacrylamide (10-15%) slab-gel electrophoresis in the presence of 0.1% of SDS (sodium dodecyl sulphate) under reducing and non-reducing conditions wherein the said proteins were visualized by silver staining (Morrissey, 1981).

The results from SDS-PAGE analysis using an acrylamide concentration gradient of 5-15% and run on the proteins purified above, indicated that all recombinant protein C's obtained from the expression in Example I (c) migrated as single bands with relative molecular masses similar to those of the respective plasma-derived proteins under non-reducing conditions. Human protein C had an apparent molecular mass of 62 KDa, whereas the molecular mass of bovine protein C was somewhat smaller. In agreement with previous reports, it was found that plasma-derived human protein C, recombinant wild-type protein C and mutant protein C exhibited two subforms corresponding to α and β protein C as glycosylation variants (Miletich and Broze, 1990). However, these two subforms were not obvious in bovine protein C. Under reducing conditions, the heavy chain from each recombinant protein C migrated as a double-band (Mr 41 KDa). A light chain (Mr 21 KDa) was also observed. This indicates that the transformed cells from Example 1(b) produce recombinant wild-type and mutant protein C derivatives in a similar manner.

(e) Characterization of Protein C Mutants

(1) To characterize the protein C mutants obtained in the previous steps, mutant and wild-type protein C were activated and their activity measured in accordance with the following test methods. Activity inhibition tests, as disclosed below, were also performed.

(1) (i) Activation of Protein C and Amidolytic Activity Assay

Activation of protein C to activated form (activated protein C, APC) by thrombin was performed as described previously (Solymoss et al., 1988) except for slight modifications. In brief, the protein C was incubated with α-thrombin (1:10, w/w) at 37° C. for 2 hrs in TBS in the presence of 5 mM EDTA. After incubation, the mixture was passed through a sulfopropyl-Sepharose column to remove thrombin. It was confirmed by the mobility difference between reduced protein C and APC on SDS-PAGE, that protein C was fully activated. The amidolytic activity of APC was measured by determination of the hydrolysis of a synthetic substrate, S2238 (Chromogenix AB, Sweden), which process was monitored at 405 nm at room temperature in a Vmax kinetic microplate reader (Victor, Molecular Devices Corp., USA).

(1) (ii) Activated Partial Thromboplastin Time (APTT) Assay

Quantitative determination of APC activity was based on the prolongation of APT time. Coatest APC Resistance kit (Chromogenix AB, Mölndal, Sweden) was used for APTT assay of APC. Fifty μl of human or bovine citrated normal plasma was incubated with 50 μl of APTT reagent at 37° C. for 200 sec, and then 100 μl of CaCl₂ (12.5 mM) containing APC (final concentrations (of 0-10 nM) were added. The clotting time was measured using an Amelung-Coagulometer KC 10 (Swedish Labex AB). All dilutions were made in TBS buffer in the presence of 0.1% bovine serum albumin (BSA).

(1) (iii) FVIIIa Inactivation Assay

Different concentrations of human or bovine recombinant APC's (0-32 nM) were mixed with protein S (20 nM) and factor V (20 nM) in microtiter plate wells (Linbro, Flow Laboratories) with a final volume of 25 μl in 50 mM Tris-HCl, 150 mM NaCl buffer containing 10.5 mM CaCl₂, 0.1% BSA, pH 7.4. Eighty μl of factor VIIIa reagent (containing bovine factor IXa, human factor VIIIa, CaCl₂ and phospholipids) were added to the mixture. After 5 min of incubation at room temperature, bovine factor X was added. The amount of activated factor X subsequently formed was measured by addition of 50 μl of a synthetic substrate S-2222 after 5 min of incubation. The reaction was stopped by adding 50 μl of 20% acetic acid after 5 min of incubation in dark at room temperature and the absorbence at 405 nm was monitored. The production of factor Xa is linearly correlated to the activity of factor VIIIa, which is expressed as percent of activity of respective control (Shen and Dahlbäck, 1994). All reagent concentrations given above are final concentrations.

(1) (iv) Prothrombin Time (PT) Assay

The inactivation of factor V by APC was measured according to the PT assay. One hundred μl of human or bovine plasma (1:3 dilution) were incubated at 37° C. for 120 sec, whereafter clotting was initiated by adding 300 μl of a mixture of Neoplastin and APC (Neoplastin: APC, 2:1, v/v). The final concentrations of APC were from 0 to 30 nM. The assay was performed on an Amelung-Coagulometer KC 10.

(1) (v) Inactivation of Protein C and Protein C Mutants In Human Plasma APC derived from activation of protein C, either human or bovine wild-type or mutants thereof, were diluted to 70 nM with 300 μl of citrated human plasma at 37° C. Samples (40 μl) were collected and diluted 5-fold in cold TBS at points of time in a range of 0 to 60 minutes. From each diluted sample, 60 μl were added to 501 μl of a synthetic substrate S-2238 (Chromogenix AB, Sweden) (1 mM) in wells on a microtiter plate. The rate of amidolysis of S-2238 by APC was recorded continuously for 0-10 min at 405 nm (Holly and Foster, 1994).

(1) (vi) Inactivation of Protein C and Mutants Thereof by α1AT

Wild-type or mutated human APC or bovine APC (170 nM of each) were incubated separately with human α1AT (0-16 μM) in 80 μl TBS buffer containing 0.1% BSA at 37° C. overnight (Holly and Foster, 1994). Samples (20 μl) were collected and added to 100 μl of S-2238 (1 mM) in wells on a microtiter plate. The rate of hydrolysis of S-2238 was monitored at 405 nm at room temperature for 0-10 min in a Vmax kinetic plate reader.

(1) (vii) Inactivation of Protein C and Protein C Mutants by PCI

Various recombinant APC's (40 nM) were incubated with 88 nM of PCI in 1 ml TBS buffer containing 0.1% BSA at 37° C. After incubation, samples (50 μl) were collected and placed on ice at points of time ranging from 0 to 120 min, and then added to 50 μl S-2238 (1 mM). The rate of hydrolysis of S-2238 was measured from 0-10 min at 405 nm at room temperature.

(2) The results from activity tests performed as disclosed above are summarized below.

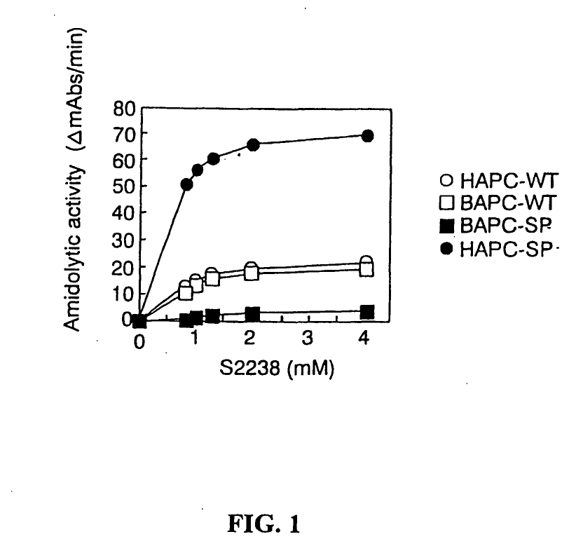

(2) (i) After activation of the protein C's from Example I (d), SDS-PAGE run on the activated protein C's indicated that the molecular masses of all recombinant wild-type and mutant APC's were similar to the corresponding plasma-derived APC, but smaller than the respective inactive forms. No intact protein C bands were observed in the APC samples, and the purity of all these proteins were more than 90% on the gel. The amidolytic activity of all APC's were measured with the synthetic substrate S-2238. For wild-type human and bovine APC the initial rate was essentially the same, whereas the initial rate for the mutant recombinant human activated protein C (designated human APC-SP) was approximately 5-fold higher than for wild-type APC. However, for the mutant recombinant bovine activated protein C (designated bovine APC-SP), the initial rate was only about 1/10 of wild-type APC. These results are shown in FIG. 1.

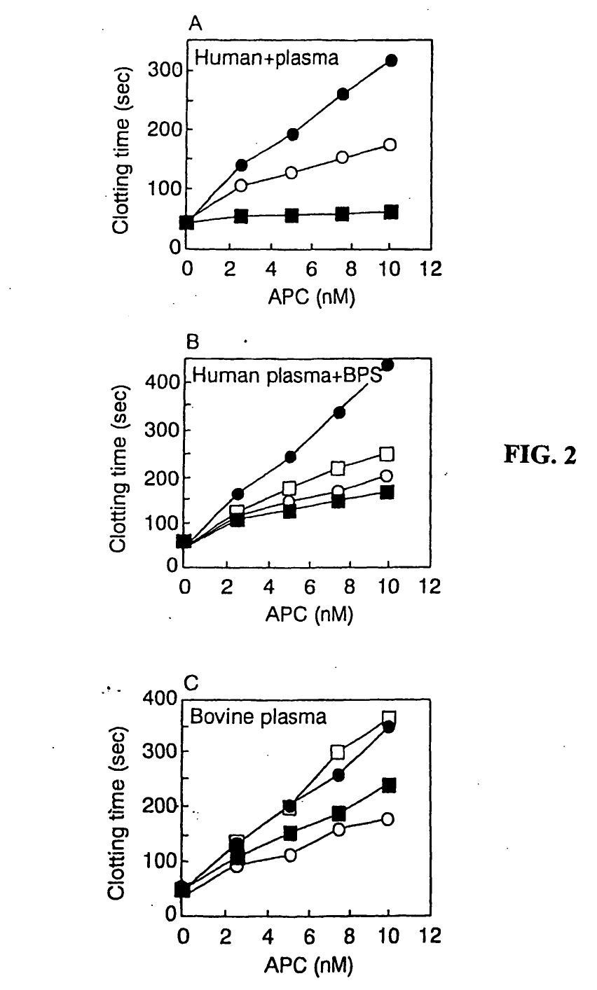

(2) (ii) In the APTT assay, the anticoagulant activity of recombinant wild-type and mutant APC's was analyzed in human plasma, in human plasma supplemented with bovine protein S and in bovine plasma. As is obvious from FIG. 2A, in human plasma, human APC-SP expressed a higher anticoagulant activity than wild-type human APC, whereas neither wild-type APC nor bovine APC-SP expressed any substantial anticoagulant activity. On the other hand, all these APC's expressed anticoagulant function in bovine plasma and in human plasma supplemented with bovine protein S. However, bovine APC and human APC-SP showed a higher anticoagulant activity than human APC and bovine APC-SP (FIG. 2B, 2C).

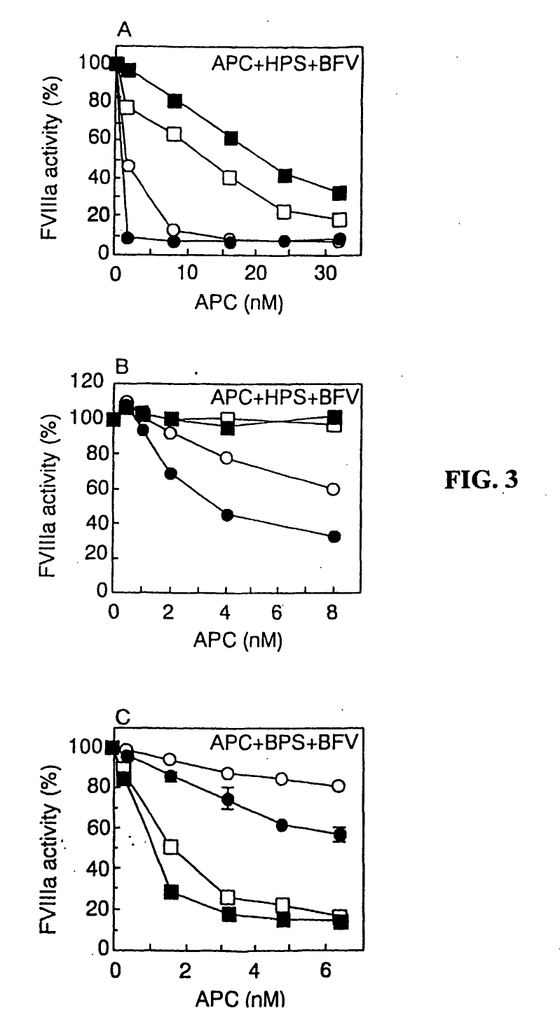

(2) (iii) In the Factor VIIIa Inactivation Assay performed in the presence of human protein S and factor V, the activity of factor VIIIa was inactivated by all APC's from section (2)(i) above but high concentrations were needed. At low concentrations, neither bovine wild-type APC nor bovine APC-SP could inactivate factor VIIIa. Human APC-SP expressed more potent anticoagulant activity than that of wild type human APC (FIG. 3A, 3B). Wild-type human and bovine APC as well as the mutants thereof were able to inhibit factor VIIIa activity in the presence of bovine protein S and bovine factor V, but both wild-type bovine APC and bovine APC-SP worked more efficiently than wild-type human APC and human APC-SP (FIG. 3C).

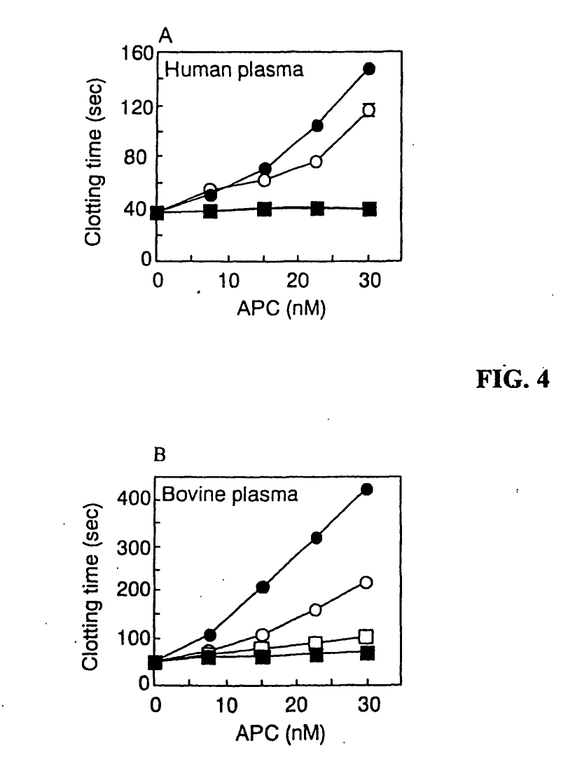

(2) (iv) In accordance with the PT assay of (1)(iv), inactivation of factor Va by the wild-type APC's and the mutants thereof was tested in human plasma and bovine plasma. Both wild-type human APC and human APC-SP increased clotting times essentially in this PT assay. Moreover, human APC-SP was more active than wild-type human APC. Neither wild-type bovine APC nor bovine APC-SP had any effect in human plasma (FIG. 4A). As is obvious from FIG. 4B, wild-type human APC and human APC-SP efficiently prolong clotting time in bovine plasma, whereas wild-type bovine APC and its mutant expressed only weak anticoagulant acitivity in bovine plasma (FIG. 4B).

(2) (v)-(vii) Results from APC inactivation tests.

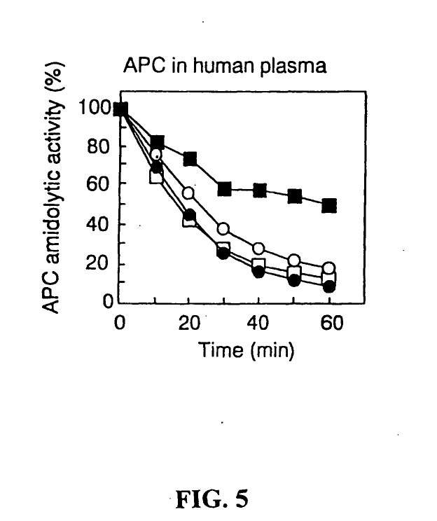

The above APC inactivation test (I)(v) showed that the amidolytic activity of wild-type and mutant APC's declined with about 60 to 90% from 0 to 60 min (FIG. 5). Thus, these APC's should be inactivated by some serine protease inhibitors, such as PCI, α1AT, α₂-macroglobulin, etc.

Indeed, both wild-type human APC and human APC-SP were substantially inhibited by high concentrations of α1AT in test (1)(vi). However, wild-type bovine APC and bovine APC-SP were almost completely resistant to the inhibition.

Test results obtained in accordance with (1)(vii) showed that bovine wild-type APC was efficiently degraded by human PCI, whereas bovine APC-SP was less efficiently inhibited by human PCI. On the other hand, the amidolytic activity of human APC-SP declined much faster than for wild-type human APC but at a rate similar to the rate for wild-type bovine APC.

### Example 2

### Preparation of Gla-Domain Mutants of Protein C

(a) Site Directed Mutagenesis

Various protein C variants containing modifications in their Gla-domains were created with recombinant technologies essentially as described previously by Shen et al (J Biol Chem 1998, 273: 31086-31091 and in Biochemistry 1997, 36 16025-16031).

A full-length human protein C cDNA clone, which was a generous gift from Dr. Johan Stenflo (Dept. of Clinical Chemistry, University Hospital, Malmö, Sweden), was digested with the restriction enzymes HindIII and XbaI and the resultant restriction fragment comprising the complete PC coding region, that is full length protein C cDNA, was cloned into a HindIII and XbaI digested expression vector pRc/CMV.

The resultant expression vector containing the coding sequence for wild-type human protein C was used for site-directed mutagenesis of the Gla-module of protein C, wherein a PCR procedure for amplification of target DNA was performed as described previously (Shen et al., supra).

Mutagenesis primers were designed for use in this procedure to cause replacement of the wild-type amino acid residues at positions 10, 11, 12, 23, 32, 33, and 44 with various other amino acids. More specifically, at position 10, histidine (H) was replaced with glutamine (Q); at position 11, serine (S) was replaced with glycine (G); at position 12, serine was replaced with asparagine (N); at position 23, aspartic acid (D) was replaced with serine (S); at position 32, glutamine (Q) was replaced with glutamic acid (E), which in the mature protein will be converted to a Gla (gamma-carboxy glutamic acid); at position 33, asparagine (N) was replaced with an aspartic acid (D); and finally at position 44, histidine (H) was replaced with a tyrosine (Y). These primers were used to produce the following variants (or mutants):
Mutant 1) designated QGN (positions 10, 11, 12 were mutated).
Mutant 2) designated SED (positions 23, 32, and 33 were mutated).
Mutant 3) designated SEDY (positions 23, 32, 33, and 44 were mutated).
Mutant 4) designated QGNSEDY, which is a combination of mutants 1) and 3) (QGN and SEDY).
Mutant 5) designated GNED and mutant 6) designated QGED (both previously described by Shen et al) were used as comparison.

To create the QGN mutant, the two following oligonucleotides were synthesized and used in the first PCR procedure, viz. primer A having the nucleotide sequence: 5′-AAA TTA ATA CGA CTC ACT ATA GGG AGA CCC AAG CTT-3′ (SEQ ID NO:34) (corresponding to sense of nucleotides 860-895 in the vector pRc/CMV including the Hind III cloning site) and primer B having the nucleotide sequence: GCA CTC CCG CTC CAG GTT GCC TTG ACG GAG CTC CTC CAG GAA (SEQ ID NO: 47) (corresponds to the second strand of the DNA stretch that encodes amino acids 4-17 with positions 10-12 mutated, which is shown by the underlining of the corresponding nucleotides). These primers A and B were used in the PCR reaction wherein wt human protein C cDNA was used as template. The PCR product was cleaved with Hind III and Bsr BI that yielded an approximately 200 bp long fragment containing the mutant amino acid residues. This fragment was ligated to two other DNA pieces, one being a Bsr BI-Xba I fragment encoding a large part of wt human protein C cDNA and the other being the Hind III-Xba I cleaved pRc/CMV vector. The ligated cDNA was checked with restriction enzyme cleavage (Hind III/Bsr BI) and sequencing to confirm the QGN mutations.

Several steps were made to create the SEDY. The first was to create the S23 mutation in a cDNA that had already the E32D33 mutation (Shen et al J Biol Chem 1998, 273: 31086-31091). Two primers were made for the S23 mutation, one being designated primer C and the other being designated primer D. Primer C had the nucleotide sequence: ATA GAG GAG ATC TGT AGC TTC GAG GAG GCC AAG (SEQ ID NO:48) (mutation is underlined); and primer D had the nucleotide sequence: CTT GGC CTC CTC GAA GCT ACA GAT CTC CTC TAT (SEQ ID NO: 49) (mutation is underlined). To create mutant cDNA, two PCR reactions were performed wherein mutant cDNA ED was used as a template and wherein primers A and C were used in the first reaction whereas primers D and E were used in the second reaction. Primer E had the nucleotide sequence: 5′-GCA TTT AGG TGA CAC TAT AGA ATA GGG CCC TCT AGA-3′ (SEQ ID NO:37) (antisense to nucleotides 984-1019 in the vector pRc/CMV including the Xba I cloning site). The first PCR reaction that involved primers A and C amplified the 5′ part of the protein C cDNA (encoding up to amino acid 28), whereas the second PCR reaction that involved primers D and E generated the 3′ part of the cDNA encoding from amino acid 18 until the end of the protein C. The two products produced in these reactions were then combined in a further PCR reaction wherein primers A and E were used. The final product from this procedure was a cDNA encoding the whole protein C carrying mutations at positions 23, 32 and 33. Then, the PCR product was cleaved with Hind III and Sal I, which gave a 360 bp 5′ fragment that was purified and ligated with the Sal I-Xba I fragment of wt protein C into the Hind III-Xba I cleaved pRc/CMV vector. This vector thus contained cDNA for the full-length mutant SED. This cDNA was used as template in a PCR reaction to create the mutant SEDY, i.e. position 44 was mutated from histidine to a tyrosine (Y). In this reaction, primer A was combined with a primer F designed to mutate position 44 and having the following nucleotide sequence: CTG GTC ACC GTC GAC GTA CTT GGA CCA GAA GGC CAG (SEQ ID NO:50) (corresponds to the second strand encoding amino acid residues 39-49—the underlined codon being the mutation spot). The PCR product was cleaved with Hind III and Sal I and the about 360 bp long fragment was ligated to the remaining part of the protein C cDNA, i.e. the Sal I-Xba I fragment and the Hind III-Xba I cleaved pRc/CMV.

The fully mutated protein C cDNA, that encodes the mutant QGNSEDY, was then created using cDNAs for the QGN and SEDY mutants. The combination was created using restriction enzyme digestion and ligation of appropriate fragments. Thus, the QGN variant cDNA was cleaved with Hind III and Bsr BI and the about 200 bp long 5′ fragment was isolated and used together with the Bsr BI-Xba I fragment (about 1000 bp long) derived from the SEDY cDNA. The two fragments were ligated into Hind III-Xba I cleaved pRc/CVM to generate the full length variant protein C cDNA encoding QGNSEDY (also referred to as “ALL” in this text). The final product was tested with sequencing and found to contain the correct mutations.

For the record, the E32D33 mutant was created in a similar fashion (this mutant is described in detail in Shen et al J Biol Chem 1998, 273: 31086-31091) using the primer G: 5′-CAG TGT GTC ATC CAC ATC TTC GAA AAT TTC CTT GGC-3′ (SEQ ID NO: 51) (antisense for amino acids 27-38 with the E32D33 mutation underlined).

DNA sequencing confirmed all mutations. Cell culture in human 293 cells, expression, purification, and characterization of protein C molecules were performed as described earlier (Shen, Let al J Biol Chem 1998, 273: 31086-31091).

In brief, the resultant human protein C cDNA containing the desired mutations was digested with SacII and ApaI, and then the fragment from the SacII and ApaI digestion (nucleotides 728-1311) was cloned into the vector pUC 18 which contains intact human protein C fragments (HindIII-SacII, 5′ end-nucleotide 728; and ApaI-XbaI, nucleotide 1311-3′ end) to produce human protein C full length cDNA comprising the desired mutations, viz. coding for a human protein C mutant comprising the mutated sequence instead of the human wild-type sequence.

Then, each of the above mutated human protein C cDNAs was digested with HindIII and XbaI and the appropriate restriction fragment was cloned into the vector pRc/CMV, which had been digested with the same restriction enzymes. The vectors obtained were used for expression of mutated human protein C in eukaryotic cells.

Before transfection of the appropriate host cells, all mutations were confirmed by DNA sequencing by the dideoxy chain termination method of Sanger et al., supra.

(b) Production of Stable Transformants Producing Variant or Wild-Type Protein C.

To produce stable transformants producing variant or wild-type protein C, adenovirus-transfected human kidney cell line 293, was grown in DMEM medium containing 10% of fetal calf serum, 2 mM L-glutamine, 100 U/ml penicillin, 100 U/ml streptomycin and 10 μg/ml vitamin K₁, and transfected with an expression vector comprising wild-type or mutagenized protein C cDNA from step (a). The transfection was performed in accordance with the Lipofectin method as described earlier (Felgner et al., 1987). In brief, 2 μg of vector DNA that was diluted to 100 μl with DMEM containing 2 mM of L-glutamine was mixed with 10 μl Lipofectin (1 μg/μl) which was diluted to 100 μl with the same buffer. The mixture was kept at room temperature for 10-15 min and was diluted to 1.8 ml with the medium, and then added to the cells (25-50% confluence in a 5-cm Petri dish) that had been washed twice with the same medium.

(c) Expression of Variant or Wild-Type Protein C.

The transfected cells from (b) were incubated for 16 hours, whereafter the medium was replaced with complete medium containing 10% calf serum and the cells were incubated for additional 48-72 hrs. The cells were then trypsinized and seeded into 10-cm dishes contaning selection medium (DMEM comprising 10% serum, 400 μg/ml G418, 2 mM L-glutamine. 100 U/ml penicillin, 100 U/ml streptomycin and 10 μg/ml vitamin K₁) (Grinnell, et al. 1990). G418-resistant colonies were obtained after 3-5 weeks selection. From each DNA transfection procedure, 24 colonies were selected and grown until confluence. All colonies were screened by dot-blot assays using monoclonal antibody HPC₄ (specific for human protein C) to examine the protein C expression. High expression cell colonies were selected and grown until confluence in the selection medium. Thereafter, these cells were grown in a condition medium (selection medium lacking serum) to initiate expression of protein C or a variant thereof, which medium, like the selection medium was replaced every 72 h. After a suitable time period, the condition medium containing the respective expression product was collected for purification of said product in section (d) below.

(d) Purification of Recombinant Wild-Type and Mutated Proteins

Culture medium obtained in section (c) from transformants producing human wild-type or mutant protein C was subjected to a simple and convenient purification method comprising a chromatographic method termed “pseudo-affinity” and described earlier (Yan et al., Biotechnology 1990, Vol. 8, 665-61).

The purified proteins obtained above were concentrated on YM 10 filters (Amicon), dialyzed against TBS buffer (50 mM Tris-HCl and 150 mM NaCl, pH 7.4) for 12 hrs and stored at −80° C. until use thereof.

The purity and homogeneity of the above wild-type and mutant protein C's were established by SDS-PAGE. This electrophoresis procedure was run as a polyacrylamide (10-15%) slab-gel electrophoresis in the presence of 0.1% of SDS (sodium dodecyl sulphate) under reducing and non-reducing conditions wherein the said proteins were visualized by silver staining (Morrissey, 1981).

### Example 3

### Characterization of Gla-Domain Mutants of Protein C

To characterize the protein C mutants obtained in the previous steps, mutant and wild-type protein C's were activated and their anticoagulant activity was tested in different experimental systems, including plasma-based assays and set ups with purified components.

Two plasma systems were tested, one being the activated partial thromboplastin time (APTT) system and the other being the thromboplastin (TP) system. In both the APTT and the TP systems, the anticoagulant activity of increasing concentrations of wt or mutant APCs was tested. In the APTT system, the anticoagulant activity of APC is dependent both on FVIIIa and FVa degradation, whereas the TP system is mainly sensitive to FVa degradation. However, the diluted TP system is to some extent sensitive also to degradation of FVIIIa.

(a). Inhibition of Clotting by APC Variants as Monitored by an APTT Reaction.

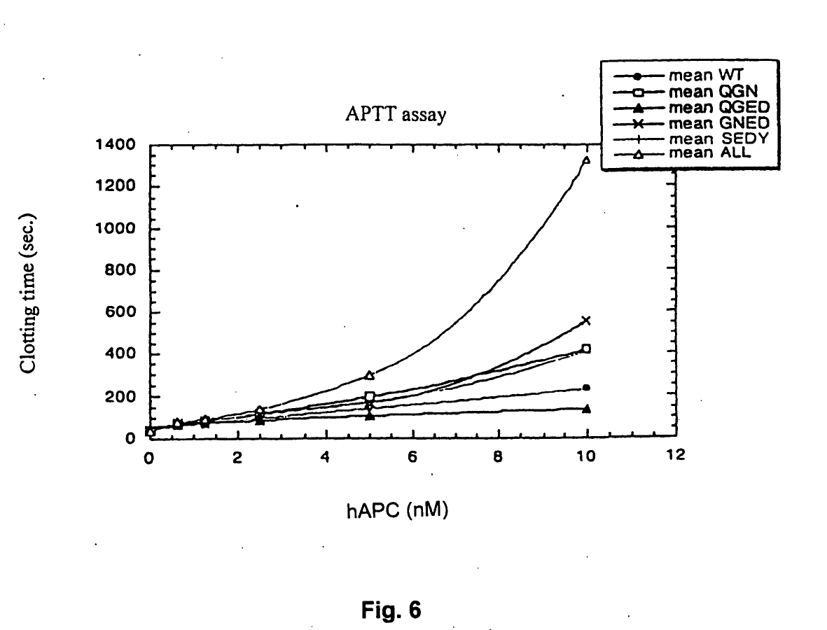

(i) Method: Plasma (50 μl) was mixed with 50 μl APTT reagent (APTT Platelin LS from Organon Technica) and incubated for 200 seconds at 37° C. Coagulation was initiated with a mixture of 50 μl APC (final concentration given in FIG. 6) and 50 μl 25 mM CaCl₂. The clotting time was measured in an Amelung coagulometer.

(ii) Results: In this APTT-based assay, the activity of wt APC was compared with the activity of the mutants 1), 3), and 4), i.e. QGN, SEDY, and QGNSEDY (ALL), as well as with the activity of two mutants previously described by Shen et al (J Biol Chem 1998, 273: 31086-31091), i.e. mutants 5) and 6) designated GNED and QGED, respectively.

With reference to FIG. 6, it is evident that the anticoagulant activity of ALL is considerably enhanced in comparison to the anticoagulant activity of wt APC. At the highest concentration used, ALL yielded clotting times exceeding 1000 seconds, whereas wt APC only gave a clotting time of about 200 seconds. The basal normal clotting time without added APC is about 30-45 seconds. On the other hand, the two previously described mutants QGED and GNED gave very different results. GNED was considerably more active than wt APC, whereas QGED in fact was less active than wt APC. The variants QGN and SEDY were equally active as GNED but were less active than ALL.

In this APTT assay, the reagents were standard commercial reagents, which stands in contrast to the reagents used in the study by Shen et al. (J Biol Chem 1998, 273: 31086-31091). In that study, a diluted APTT regent was used, since otherwise the APC variants were not more active anticoagulants than wt APC. In the discussion section of the Shen et al reference, this was explained to be due to the level of phospholipid in the reagents. If high levels of phospholipid were used, it was not easy to notice the increased activity of the APC variants used in the study by Shen et al. Only when diluted regents were used, the authors could demonstrate a strong increase in the anticoagulant activity of the APC variants.

The present variant QGNSEDY (ALL) appears to be unique as it is evidently much more active than wt APC also at standard levels of phospholipid.

(b) Impact of Human Protein S in an APTT Assay

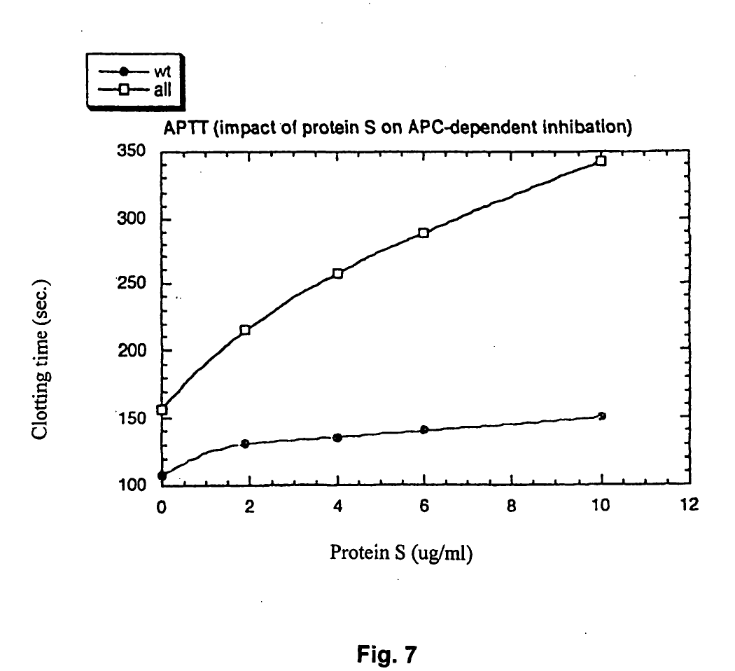

(i) Method: Increasing concentrations of protein S were added to protein S deficient plasma to obtain the final concentrations indicated in FIG. 7. Plasma aliquots (50 μl) were mixed with the APTT reagent and then incubated for 200 seconds at 37° C. Thereafter, APC, either wt or the ALL mutant (QGNSEDY), was added in a volume of 50 μl (concentration 20 nM) and clotting was then immediately initiated by the addition of 50 μl of 25 mM CaCl₂. The results are shown in FIG. 7 as clotting times plotted versus the concentration of protein S in the protein S deficient plasma.

These experiments were performed essentially as described above with reference to FIG. 6, protein S deficient plasma being used instead of the normal plasma. This protein S deficient plasma was of human origin and the protein S depletion was the result of immune-absorption using a highly efficient monoclonal antibody against human protein S(HPS54-described by Dahlbäck et al. (J Biol Chem 1990 265: 8127-35).

(ii) Results: With reference to FIG. 7, it is evident that a preferred Gla-domain variant, viz. the QGNSEDY variant, was considerably more active than wt APC also when protein S depleted plasma was used. Of particular interest is the observation, that the addition of exogenous protein S enhanced the anticoagulant activity of QGNSEDY as well as of wt APC. In absence of protein S, the mutant ALL yielded a clotting time of about 160 seconds and this clotting time was prolonged up to 350 seconds by the addition of protein S in the test system. Corresponding values obtained with wt APC were a basal clotting time of about 100 seconds in the absence of protein S and a prolonged clotting time of 150 seconds in the presence of the highest protein S concentration used in this test. Thus, it is obvious that ALL is essentially more active than wt APC both in presence and absence of protein S and that ALL moreover is potentiated by the presence of protein S. This is in contrast to the results obtained by Esmon and Smimov with their APC variants (described in WO 98/20118) that were not stimulated by protein S. Evidently, the present variant QGNSEDY is superior to the variants disclosed by Esmon and Smirnov, since it is stimulated by protein S.

(c) Inhibition of Clotting by APC Variants as Monitored by a TP System

(i) Method: Normal plasma (50 μl) was mixed with increasing concentrations of the various APC variants (50 μl aliquots whereafter clotting was initiated by the addition of thromboplastin, diluted 1/50, as a source of tissue factor. To initiate clotting, the diluted thromboplastin also contained 25 mM CaCl₂.

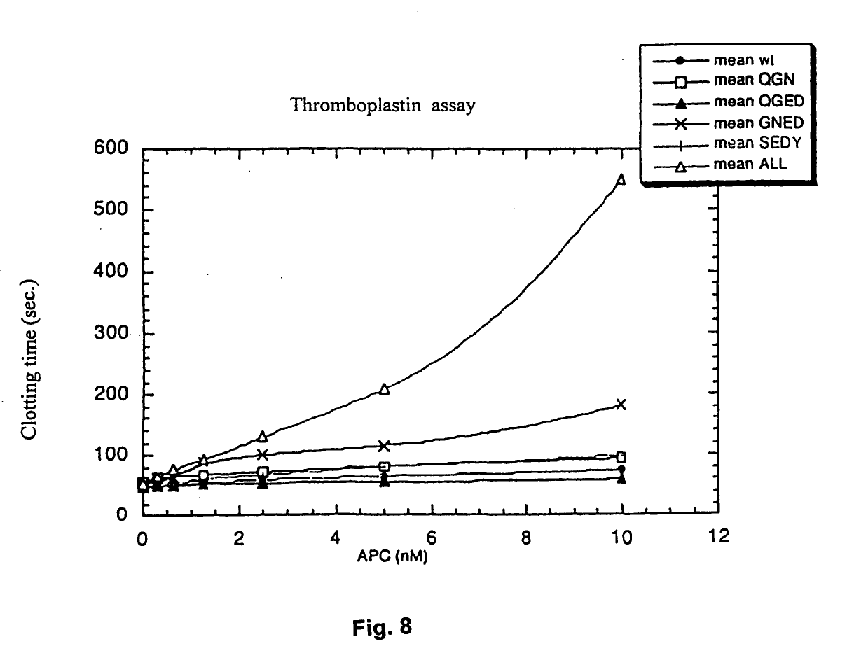

(ii) Results: As is evident from FIG. 8, the results obtained with this assay were similar to those obtained with the APTT system. Thus, the variant QGNSEDY was considerably more active than wt APC. More specifically, at the highest concentration used, the variant QGNSEDY (designated ALL in FIG. 8) yielded a clotting time that was close to 600 seconds. The second best variant was GNED, which at the highest concentration yielded a clotting time of approximately 180 seconds. In contrast, wt APC only yielded clotting times of about 70 seconds. The basal clotting time obtained without addition of exogenous APC was approximately 40 seconds.

Apparently, the results of this experiment suggest that as compared to wt APC the variant QGNSEDY has unique properties, since wt APC never exhibits an anticoagulant activity as high as the anticoagulant activity of the variant QGNSEDY, not even at increasing concentrations of wt APC. This might suggest that by mutagenesis performed to produce the Gla-domain of the variant QGNSEDY, a molecule has been created that exhibits new and distinct functions as compared to wt APC. One such function could be related to the protection of the Arg506 site in FVa that is provided by FXa. It is known that FXa binds to FVa at a site close to Arg506 and that this results in protection of the Arg506 site. Possibly, the unique and high phospho-lipid binding ability of QGNSEDY abrogates the protection provided by FXa. During the clotting assays, a certain amount of FXa is formed and this may restrict the ability of wt APC to cleave the Arg506 site in FVa. It is possible that the QGNSEDY variant could displace the FXa due to its high affinity not only for phospholipid membranes but also for the FVa molecule. Moreover, at the highest concentration of APC used in this test, the QGNSEDY variant is able to prolong the clotting times considerably more than wt APC is able to. This suggests that the APC variant QGNSEDY might have unique in vivo properties and may be able to inhibit a clotting reaction that is already ongoing.

(d) Impact of Protein S in a PT Assay

Experiments with protein S deficient plasma like those described in Example 3(b)(i), were also performed, the thromboplastin system of Example 3(c)(i) being used. The results thereby obtained were similar to those described for the APTT system in Example 3(b)(ii). In brief, it was found that the QGNSEDY variant is active in the absence of protein S, but yet, its activity is potentiated by protein S.

### Example 4

### Inactivation of FVa by APC

In this example, the enhanced activity of the APC variant QGNSEDY was established in a system, designed to more specifically characterize the degradation of FVa and wherein the loss of FVa activity over time is demonstrated.

(i) Method: Plasma FVa (0.76 nM) (plasma was diluted 1/25 and FV contained therein was activated by the addition of thrombin—this was used as the source of FVa) was incubated with APC (0.39 nM) in the presence of 25 μM phospholipid vesicles (mixture of 10% phosphatidylserine and 90% phosphatidylcholine). The buffer was 25 mM Hepes, 0.15 M NaCl, 5 mM CaCl₂, pH 7.5, and 5 mg/ml BSA and the temperature was 37° C.

At various time points, aliquots were drawn and the remaining FVa activity was determined by a FVa assay. This assay was based on the ability of FVa to potentiate the FXa-mediated activation of prothrombin. This assay contained bovine FXa (5 nM final concentration), 50 μM phospholipid vesicles (mixture of 10% phosphatidylserine and 90% phosphatidylcholine) and 0.5 μM bovine prothrombin. The generation of thrombin was measured using the chromogenic substrate S2238 (available from Chromogenix AB).

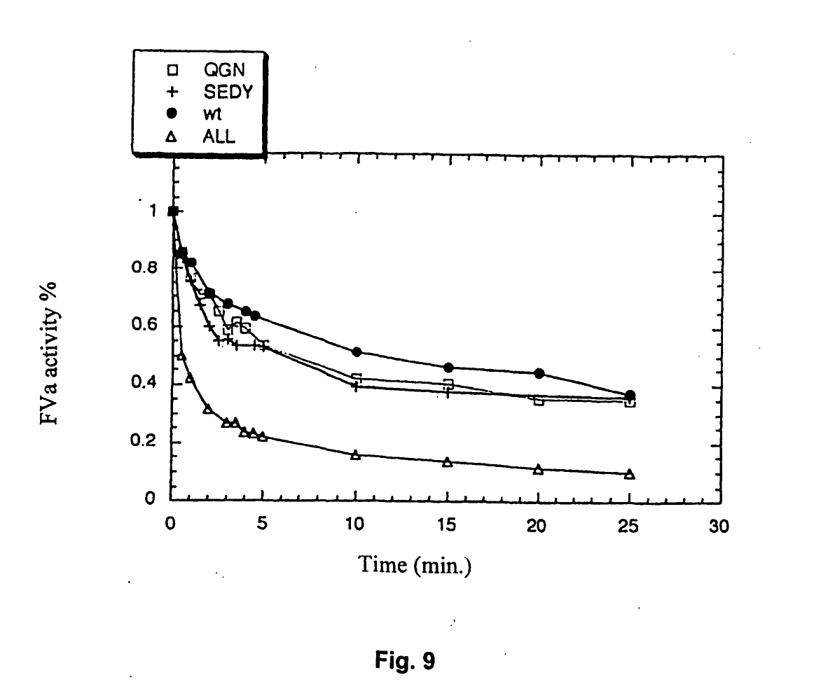

(ii) Results: The loss of FVa activity that follows upon incubation of FVa with wt APC is the result of primarily two cleavage reactions, viz. at Arg506 and at Arg306. The kinetically favored reaction is the reaction occurring at Arg506, that yields the initial rapid loss of FVa activity that is observed during the first 5 minutes of incubation. The Arg506 cleavage only results in partial inhibition of FVa because as has been shown by Nicolaes et al. (J Biol Chem 1995 270: 21158-66), FVa cleaved at Arg506 is still partially active as cofactor to FXa, about 40% of its activity being maintained. On the other hand, the slower cleavage at Arg306 results in a complete loss of FVa activity. This Arg 306 cleavage is progressing slowly as is reflected in the slow decrease in FVa activity observed between 5 minutes and 25 minutes of incubation. As is evident from FIG. 9, the variants QGN and SEDY are only slightly better than wt APC, whereas the present variant QGNSEDY is considerably more potent. The present variant QGNSEDY not only yields a very fast drop in FV activity down to approximately 20% FVa activity during the first five minutes but ultimately also inhibits FVa almost completely. These results suggest that the present variant QGNSEDY not only cleaves FVa at Arg506 faster than what is seen for wt APC, but as opposed to wt APC, also cleaves FVa at the Arg306 site.

Experiments similar to those described above (results not shown) were performed wherein the ability of the variant QGNSEDY and of wt APC to inactivate FVa was compared to this ability of the previously characterized variant GNED (cf. FIG. 6 and FIG. 8). The GNED variant was found to give a curve positioned almost exactly between the curves obtained for the other two APC:s i.e. GNED was more potent than wt APC but less efficient than the present variant QGNSEDY. These experiments were all performed without addition of exogenous protein S. The results obtained were consistent with the results of the experiments performed in Example 3(a) and (c) and illustrated in FIG. 6 and FIG. 8, respectively, that also show that the previously disclosed GNED variant has intermediate activity.

### Example 5

### Inactivation of FVa by APC

In this example, the concentration of APC was varied and the remaining FVa activity was measured after 10 minutes of incubation using the prothrombinase assay described in Example 4(i).

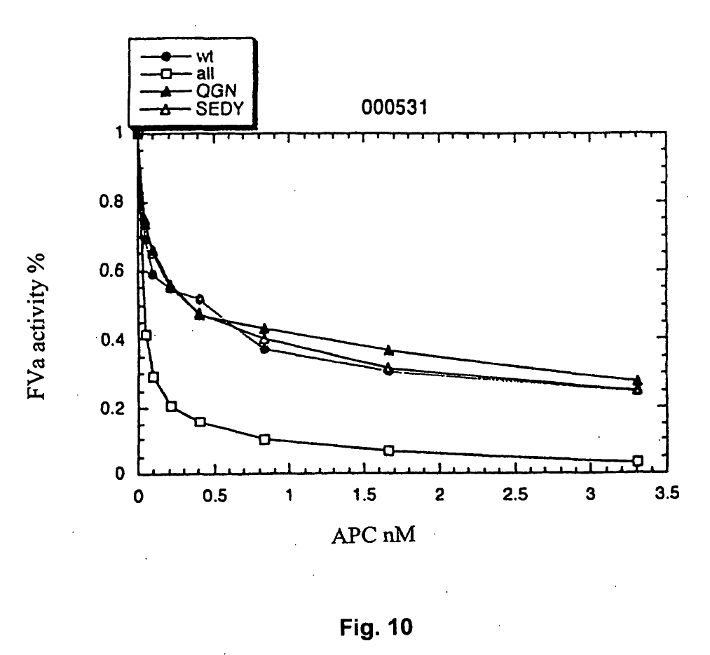

(i) Method: FVa obtained from diluted normal mixed plasma (0.76 nM) was incubated with increasing concentrations of APC (final concentrations given in FIG. 10) and 25 μM phospholipid vesicles (phosphatidylserine/phosphatidylcholine, 10/90, mol/mol) in 25 mM Hepes (pH 7.5), 150 mM NaCl, 5 mM CaCl₂ and 5 mg/ml BSA at 37° C. FVa activity was measured with the prothrombinase assay as described in Example 4(i).

(ii) Results: From FIG. 10, it is evident that these experiments clearly demonstrate the superior efficiency of the mutant ALL, i.e. the variant QGNSEDY. Even quite low concentrations of APC resulted in a potent inhibition of FVa activity. Moreover, it is obvious from the curves in FIG. 10, that the mutant ALL not only cleaves at the Arg506 site, which results in an intermediate degradation product of FVa that exhibits about 40% activity but also cleaves at the Arg306 site, which results in an almost complete loss of FVa activity.

### Example 6

### Inactivation of Normal and Q506 Mutant FVa by APC

In this example, the normal plasma FVa was replaced with FVa from APC resistant plasma (obtained from an individual with homozygosity for FV:Q506-FV Leiden). This experiment was performed both in the presence and absence of exogenous protein S.

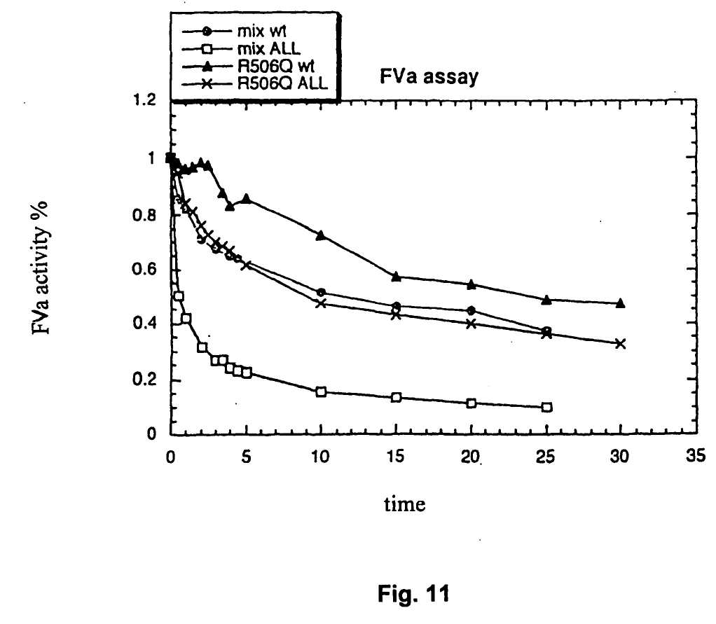

(i) Method: Plasma FVa obtained either from normal pooled plasma or from an individual with homozygous APC resistance (FV:Q506 or FV Leiden) was incubated with 0.4 nM APC and 25 μM phospholipid vesicles as described in Example (4)(i) except that purified. human protein S (100 nM) was added to ensure cleavage at Arg 306. At time points as indicated in FIG. 11, remaining FVa activity was determined.

(ii) Results: The addition of wt APC resulted in a slow decrease in FVa activity corresponding to cleavage at Arg306, the slope of the corresponding curve in FIG. 11 being similar to the second part of the curve for wt APC illustrated in FIG. 9. In contrast, the present variant QGNSEDY (or ALL) resulted in a more rapid drop in FV activity consistent with enhanced cleavage of FVa at Arg306 by the APC variant. The addition of protein S enhanced the effect both of wt APC and the QGNSEDY, but yet the difference between the two proteins remained. Thus, it can be concluded that protein S stimulates not only wt APC but also the present APC variant, the latter exhibiting a considerably enhanced binding affinity for the phospholipid. This is of interest, since it has been suggested that protein S functions by enhancing the binding affinity of APC for the phospholipid. If this would be the only mechanism by which protein S works, then one would expect that addition of protein S would decrease the difference between wt APC and the QGNSEDY variant.

### Example 7

### Membrane-Binding Affinity of APC

To investigate the ability of wt and variant protein C's to bind to phospholipid membranes, the surface plasma resonance technique was used. A commercial variant of this technique is available from BIAcore. In this example, a BIAcore 2000 was used.

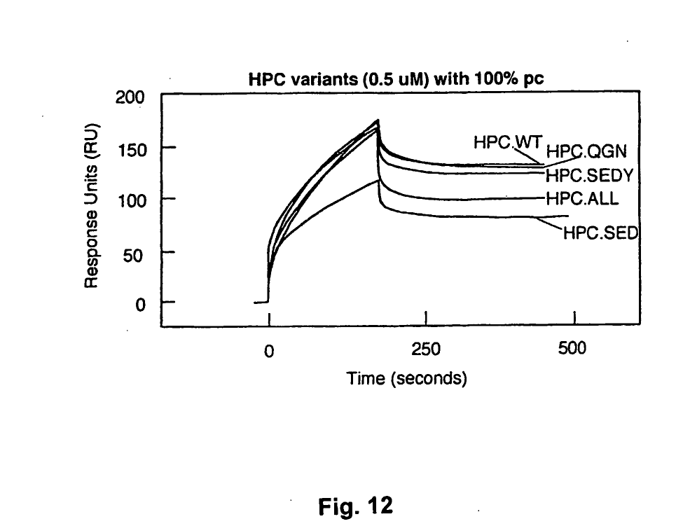

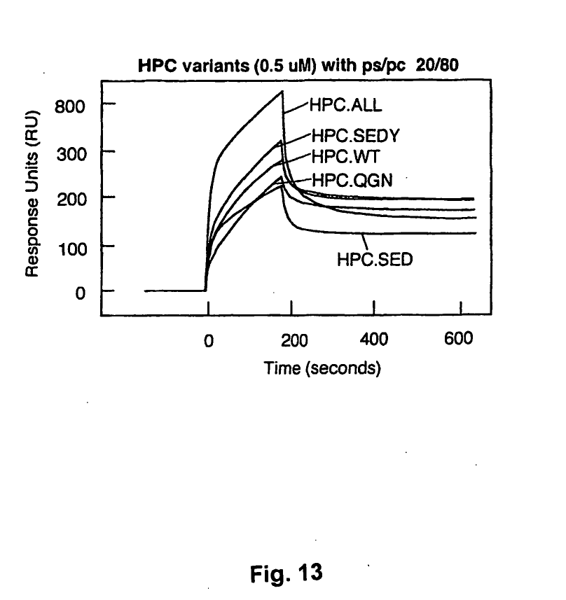

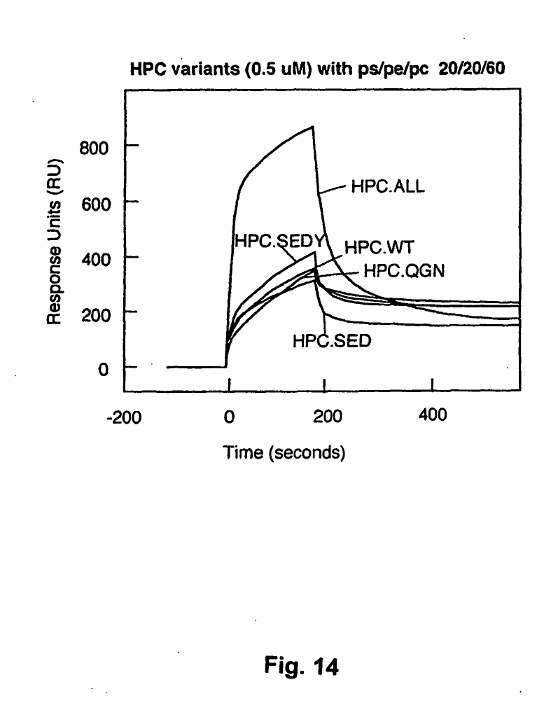

(i) Method: Phospholipid vesicles were captured on the surface of an L1 sensor chip from BIAcore. These chips consist of a dextran hydrogel with covalently coupled hydro-phobic aliphatic groups. Three different kinds of vesicles were prepared using extrusion technique (using an Avestin Lipofact basic extrusion apparatus), the three types of vesicles having different phospholipid composition, viz. 1) 100% phosphatidylcholine (FIG. 12), 2) 80% phosphatidylcholine and 20% phosphatidylserine (FIG. 13), and 3) 20% phosphatidylserine, 20% phosphatidylethanolamine and 60% phosphatidylcholine (FIG. 14). Four protein C mutants, viz. HPC ALL (i.e. QGNSEDY), SEDY, QGN and SED, and wt HPC were tested. In these experiments, the protein C concentration was 0.5 μM and the buffer used was 10 mM Hepes, 0.15 M NaCl, containing 5 mM CaCl₂, pH 7.5.

Phosphatidylcholine-containing membranes do not bind the vitamin K-dependent proteins unless the negatively charged phosphatidyl serine is part of the membrane. Phosphatidylethanolamine is of particular interest because the presence of this type of phospholipid in the membrane has been shown to enhance the binding of protein C and to enhance the rate of degradation of FVa. Thus, in this example it is investigated whether or not the protein C variants demonstrated a changed specificity for the phospholipid types. The different recombinant protein C variants were injected into the BIAcore machine, which had a chip that contained different surface areas covered by the three types of phospholipid membranes.

(ii) Results: A concentration of protein C of 0.5 μM was used since, at this concentration, wt protein C is not expected to give any particularly strong binding, because the K_{d} for protein C to negatively charged phospholipid membranes is approximately 15 μM. Thus, in these experiments it should be possible to see any increased binding ability of the protein C variants. As is evident from FIG. 12, there was very little, if any, binding of the protein C variants to the membrane containing 100% phosphatidylcholine. The maximum response units reached were only about 160. From FIG. 13, it is obvious that, on membranes containing 20% phosphatidylserine, there was considerably better binding in particular by the variant QGNSEDY (or ALL) that demonstrated a rapid association of protein C as reflected by the sharp increase in the response as plotted on the Y-axis. The other variants, i.e. QGN, SEDY and SED, behaved like wt protein C. The results shown in FIG. 14, illustrate that the most striking difference between the QGNSEDY (or ALL) variant and the wt protein C was observed when the phosphatidylethanolamine-containing membranes were used. The QGNSEDY variant demonstrated a sharp increase in binding to the membrane and very quickly reached a response of about 700 units. During the following 200 seconds, the response rose to approximately 850 response units. The dissociation was followed by discontinuation of the protein C infusion and the bound proteins were relatively quickly released from the membranes. The binding was calcium dependent, since EDTA reversed the binding completely. This behavior is expected from the vitamin K-dependent proteins.

### Examples 8-10

The SP-mutant and the Gla-domain mutants of protein C that have been prepared above are conveniently used as precursors to create protein C combination variants containing mutations both in the Gla- and the SP domains of protein C. This is accomplished at the cDNA level using standard DNA molecular biology methods.

Preferentially, restriction enzyme cleavages, fragment isolation and fragment ligation are used.

### Example 8

### Preparation of a combination variant

The cDNA for the individual protein C variants is present in the PcDNA3 vector and cloned into the vector using the Hind III-Xba I sites. The whole cDNA for the protein C variants can consequently be liberated from the vector by digestion with restriction enzymes Hind III and Xba I. The cDNA can be further fragmented with specific enzymes. A particularly useful enzyme for the creation of the combined variant is Sal I, which cleaves the protein C cDNA into two fragments, a small that corresponds to the 5′ part of the cDNA (first 259 nucleotides of the coding sequence), i.e. the part that encodes the N-terminus of the protein including the Gla-domain and a bigger 3′ fragment that encodes the rest of the protein C. The Sal I cleavage site is located at a position just 3′ of the codon for position 44 and therefore, the smaller fragment will encode the full Gla-domain. Combined variants having mutations in both the Gla-domain and the SP domain can be created by combining the smaller 5′ fragment from the Gla-domain mutated protein C with the larger 3′-fragment of protein C variants having mutations in the SP domain. The two protein C cDNA fragments are combined with the Hind III-Xba I cleaved PcDNA3 vector in a ligation reaction and the ligated DNA is used to transform bacteria. Antibiotic-resistant colonies are selected with standard technology and the plasmid DNA is isolated and sequenced to confirm the presence of mutations in the cDNA. The DNA is then used to transfect HEK 293 cells and recombinant protein C is expressed, purified and characterised as described for the other protein C variants.

According to this method, “super PC/APC” containing the Gla-domain mutation QGN SEDY and the SP-domain mutation comprised of the modified sequence of SEQ ID NO: 7 is prepared.

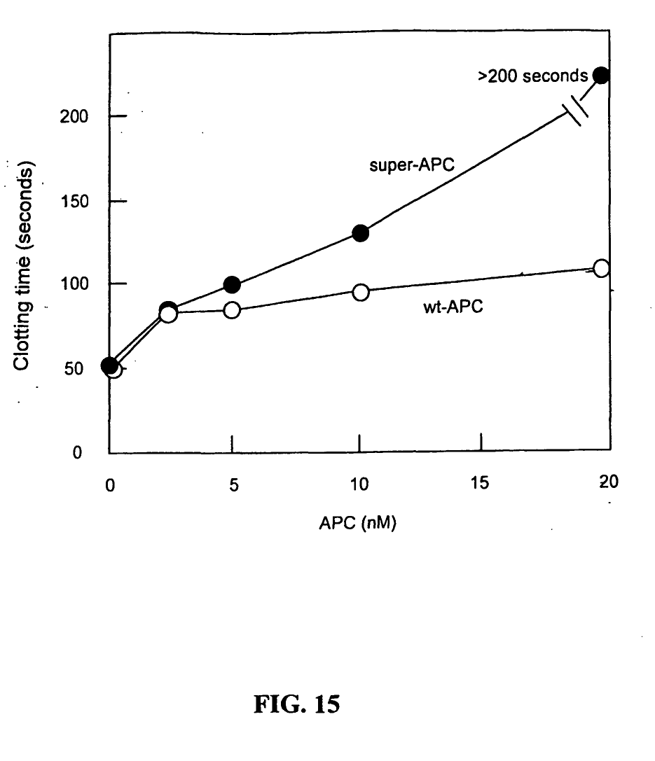

The recombinant protein C is activated by thrombin and tested in an APTT reaction. The super-APC resulted in a more pronounced prolongation of clotting times than wild-type APC (FIG. 15). In this experiment, increasing concentrations of wt- or super-APC are added to an APTT clotting reaction and the clotting time is monitored. The wt-protein C prolonged the clotting time as expected and the highest concentration tested (20 nM) yields a clotting time of around 100 seconds, which is around double of that observed in the absence of APC. The Super APC is considerably more active and already at an APC concentration of 5 nM, the clotting time is prolonged to similar levels. At higher concentrations of super APC, the clotting time is further prolonged and at 20 nM super-APC, the plasma does not clot within the 200 seconds observation time.

The APTT-test was performed in human plasma according to the following procedure.

Human citrated plasma (50 μl) was mixed with 50 μl APTT reagent. After 180 seconds incubation at 37° C., 50 μl APC was added at the concentrations indicated in FIG. 15. The APC was contained in a 50 mM Tris-HCl, 0.15 M NaCl buffer pH 7.5, also containing 30 mM CaCl₂ and 0.1% BSA (bovine serum albumin). In FIG. 15, the dots represent the mean of two determinations.

### Example 9

In this example, the effect of combining the mutation of the Gla variant called GNED with the SP mutation wherein the modified region WGYRDETKRNR (SEQ ID NO: 7) replaces the wild-type sequence from position 300 through 314, inclusive. The GNED mutant carries the mutations G11, N12, E32, and D33 in the Gla domain (this variant is described in Shen et al JBC 1998). This combination produced a protein C variant having enhanced affinity for negatively charged phospholipid membranes and the activated form of this variant demonstrated enhanced anticoagulant activity in clotting assays containing low concentrations of phospholipid, e.g. in the diluted APTT and diluted tissue factor-dependent assays as described in WO99/20767. However, in a regular APTT reaction, the GNED-APC was only as active as wt-APC or slightly better. In example 1, the SP variant has been shown to yield enhanced anticoagulant activity (at least 100% increase) but under some clotting assay conditions (certain APTT reagents), it has been difficult to clearly demonstrate the enhanced anticoagulant activity of the SP mutant. The idea was now to combine the GNED and SP mutations into one new protein C variant. As discussed above, the hypothesis was that the enhanced phospholipid-binding ability of the Gla mutations, when combined with the more efficient SP mutant would result in a protein C hybrid with significantly enhanced anticoagulant potential. Such variants could be useful as therapeutics in situations with enhanced clotting activity such as thromboembolic disorders, sepsis, etc. The new variant was denoted GNED-SP and it was created by combining cDNA encoding the Gla domain from the GNED variant with cDNA encoding the serine protease domain of the SP mutant. This was done with standard DNA technology as outlined in the method section. The mutant cDNA was then used to transfect 293 HEK cells and high expressing colonies were isolated and expanded and condition medium containing the recombinant protein was collected. The recombinant protein was purified and characterised and activated by thrombin to generate APC as described in previous sections.

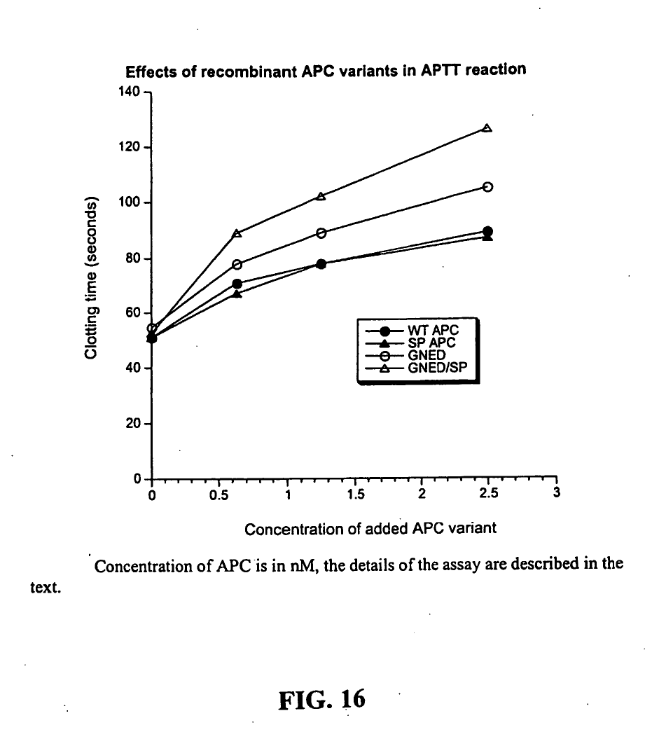

The APTT based assay was performed as follows: 50 μl human plasma from a normal individual was mixed with 50 μl APTT reagent (2 parts of Organon Platelin and 1 part of TBS buffer with 0.1% BSA) (TBS stands for 50 mM Tris-HCl, 0.15 M NaCl, pH 7.5). After 180 seconds of incubation at 37° C., 50 μl of 25 mM CaCl₂ containing increasing concentrations of APC were added and the clotting time was recorded. The results are presented in FIG. 16. In this experiment, the SP variant was equally active as wt APC, whereas the GNED-APC variant was clearly better than wt APC. However, the GNED-SP was the best and clearly more anticoagulant than any of the other tested variants.

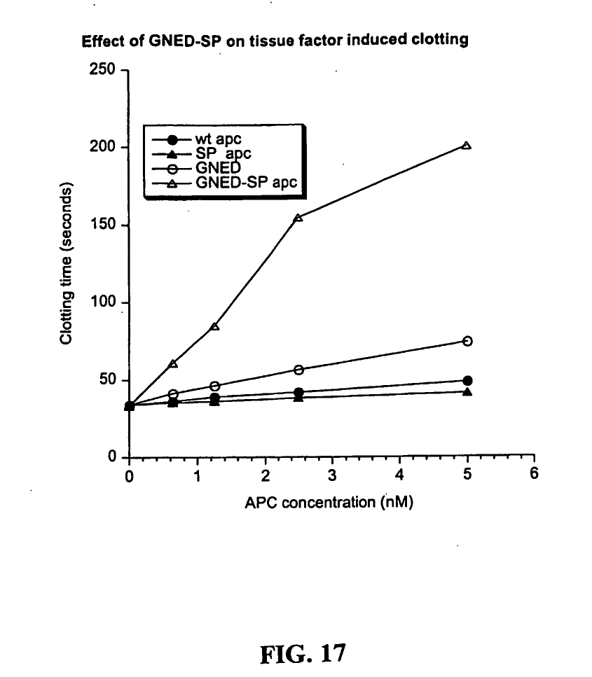

In the next assay, 50 μl human plasma from a normal individual was mixed with 100 μl diluted Simplastin (a tissue factor-containing reagent that was diluted 1:50 to give a clotting time of around 35 seconds) that contained increasing concentrations of APC as well. The SP and wt variants were approximately equally active whereas the GNED-APC was more anticoagulant than wt-APC. However, the GNED-APC variant was very efficient and already 1-2 nM GNED-SP APC resulted in distinctly prolonged clotting times. (FIG. 17)

### Example 10

In this Example, the variant of Example 8, i.e. the hybrid created by combining the GLA domain from QGNSEDY (ALL) with the SP domain of the SP mutant of Example 1, which variant is referred to as super-APC, is further tested. The super-APC was tested more extensively than GNED-SP APC, including tests using whole blood, which also involve the effect of APC on platelet supported clotting. Not only human plasma was tried but also rat and mouse plasma. The anticoagulant effects of super-APC were particularly strong in the animal plasmas. These animal plasma experiments can be taken to prove the point that the super-APC is more efficient than QGNSEDY-APC. The in vivo effects of super-APC are difficult to predict but it is likely that super-APC is more efficient as an anticoagulant than QGNSEDY-APC.

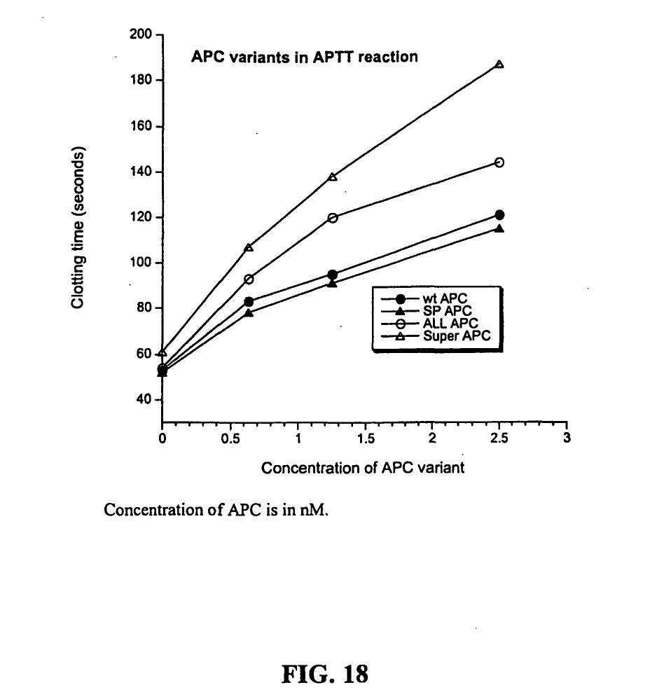

In the APTT reaction (FIG. 18) (experiment performed as described above with the same APTT reagent), the super-APC was more efficient in prolonging the APTT clotting time than any of the other variants. ALL-APC, which is the same as QGNSEDY-APC, was more efficient than wt APC and SP-APC. The super-APC was highly efficient and it is noteworthy than a distinct anticoagulant effect is observed already at less than 1 nM APC concentration.

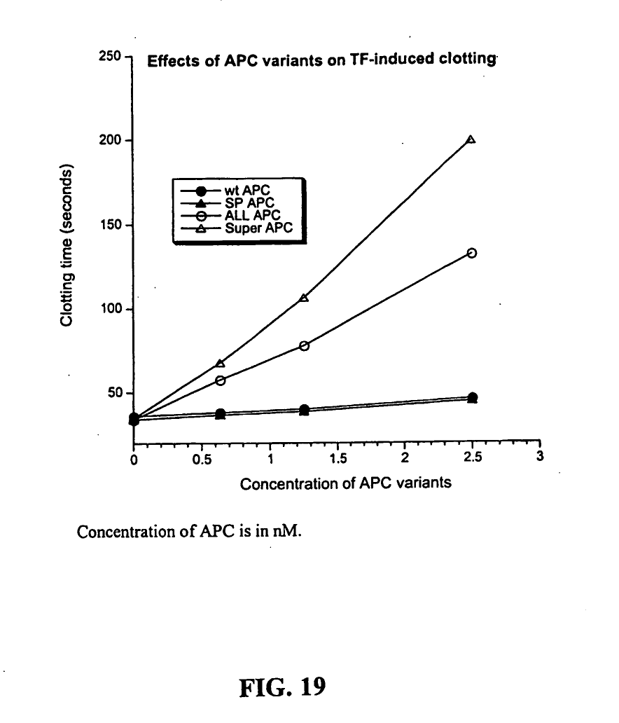

In the tissue-factor-based assay (FIG. 19), similar results were obtained, super-APC being a more potent anticoagulant than any of the other variantss. The lowest concentration of super-APC was approximately equally efficient as 10-fold higher levels of wt APC, demonstrating the high efficiency of the super-APC variant.

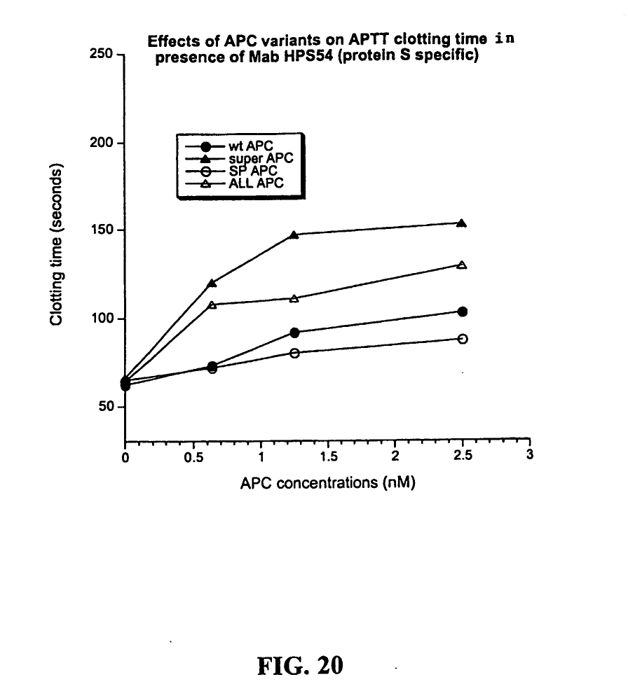

To test if the anticoagulant effect of super-APC was dependent on the presence of protein S, an additional experiment was performed in which the plasma protein S was inhibited with an excess of a monoclonal antibody denoted HPS54, which is known to be efficient in inhibiting the APC cofactor activity of protein S. In this experiment, the plasma was incubated with HPS54 (50 μg/ml final concentration) for 1 hour at room temperature, which is sufficient to inhibit protein S cofactor activity as described previously (Dahlbäck, B., Hildebrand, B., and Malm, J. Characterization of functionally important domains in vitamin K-dependent protein S using monoclonal antibodies (1990) J. Biol. Chem. 265, 8127-8135.). The plasma was then used in an APTT reaction and in this case, an APTT reagent from Chromogenix AB, Sweden, was used. In other respects, the experiment was performed as described for FIG. 16. Both wt APC and SP-APC were rather inefficient in prolonging the clotting time (FIG. 20). In contrast, both ALL-APC and super-APC effectively prolonged the clotting time already at concentrations lower than 1 nM. The effect of super-APC was stronger than ALL-APC. This experiment shows that the super-APC variant is an efficient anticoagulant even in the absence of protein S. This strong anticoagulant effect of super-APC even in the absence of protein S might be a therapeutically highly interesting feature. It is also important to note that the super-APC was stimulated by the presence of protein S.

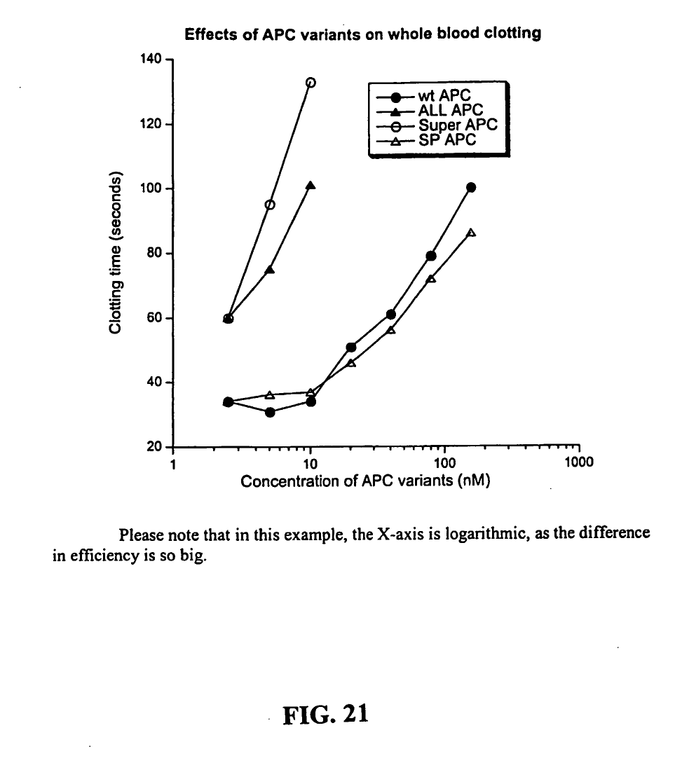

Regular APC is rather inefficient as anticoagulant in the presence of platelets. Thus, under normal physiological conditions, it is likely that APC has no, or only a weak effect on the reactions that take place on the platelet surface. As arterial thrombosis in general involves platelets, it might be interesting to obtain an APC variant that is efficient in inhibiting the clotting reactions on platelets. To test this, a whole-blood clotting test was devised, which depended on the initiation of clotting by factor Xa (FXa). Citrated whole blood (75 μl) was incubated at 37° C. for 180 seconds before the addition of 75 μl FXa (1 nM) containing 25 mM CaCl₂ and increasing concentrations of the different APC variants. The buffer was the TBS-BSA mentioned above. In the absence of APC, the clotting time was approximately 33 seconds. Addition of wt APC (FIG. 21) was rather inefficient and prolongation of clotting time was only observed at >20 nM APC. The SP-APC variant was approximately equally efficient as wt-APC. In contrast, the ALL and super variants were much more potent. In the case of ALL-APC, it is estimated that it is approximately 20 times more active than wt APC, whereas super-APC is even more active. From FIG. 21, it is estimated that the super-APC is approximately 40 times more active than wt APC in the whole-blood system.

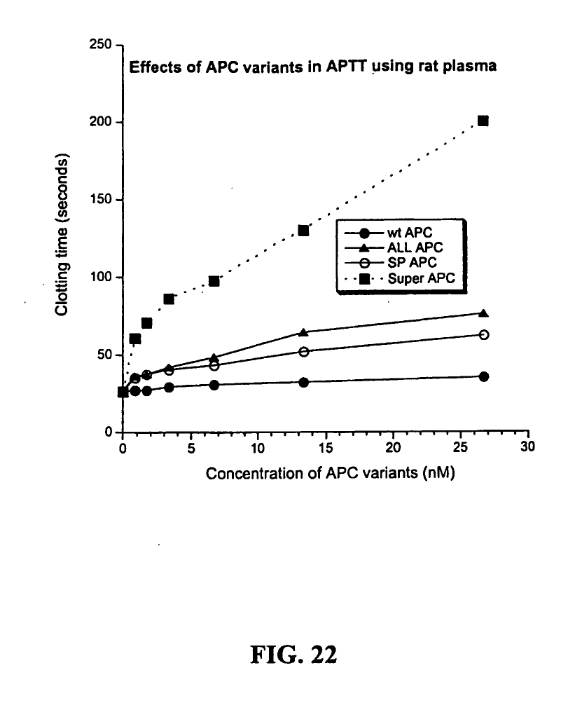

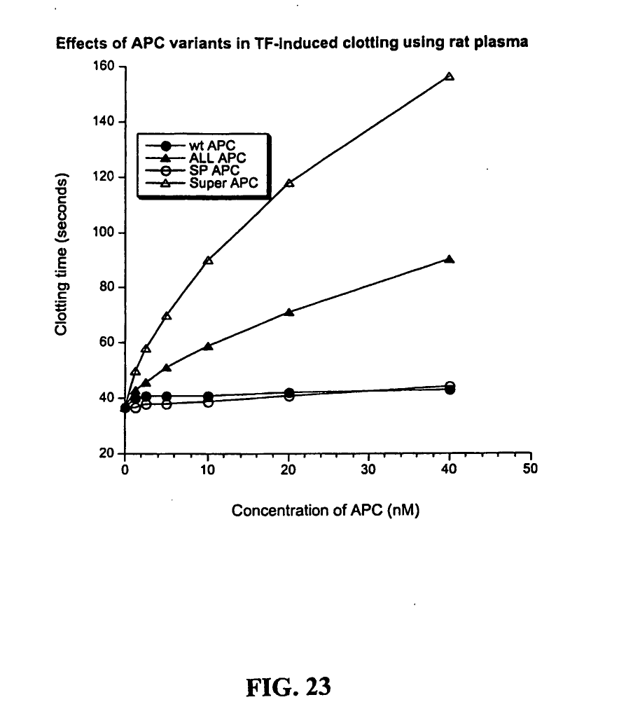

The animal plasma experiments were performed using both the APTT- and the tissue factor-based assay systems. The clotting times obtained in the APTT reaction with rat plasma were generally shorter than those observed in the human system. It was found that it was adequate to dilute the APTT reagent 1:2 with TBS-BSA to obtain reasonable clotting times of around 25 seconds (FIG. 22). The addition of wt APC was found to be inefficient in prolonging the clotting time of rat plasma. In contrast, both SP-APC and ALL-APC were effective even though the effects were modest. In contrast, the super-APC was highly efficient and even the lowest concentration tested (2.5 nM) effectively prolonged the clotting time. Even though it is based on rat plasma, this experiment demonstrates that super-APC is much better than either SP-APC or ALL-APC. The results obtained with the tissue factor-induced system yielded similar conclusions (FIG. 23). In this system, wt-APC and SP-APC were similar, whereas ALL-APC was a more potent anticoagulant. However, the super-APC was clearly much more efficient than any of the other variants.

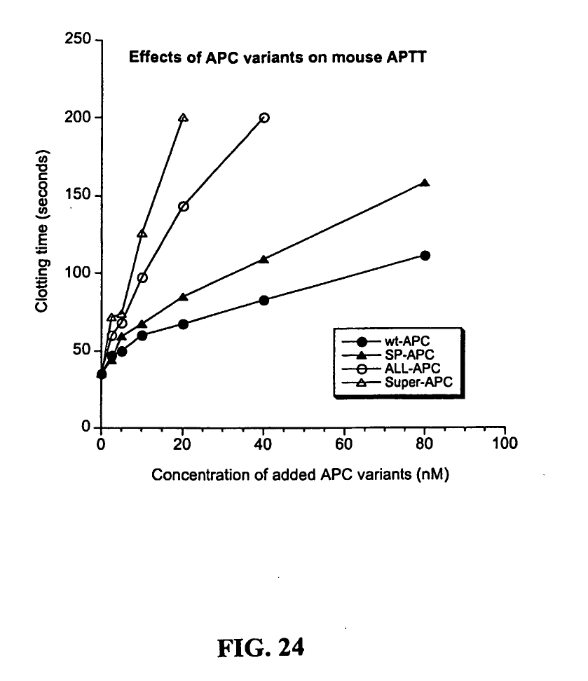

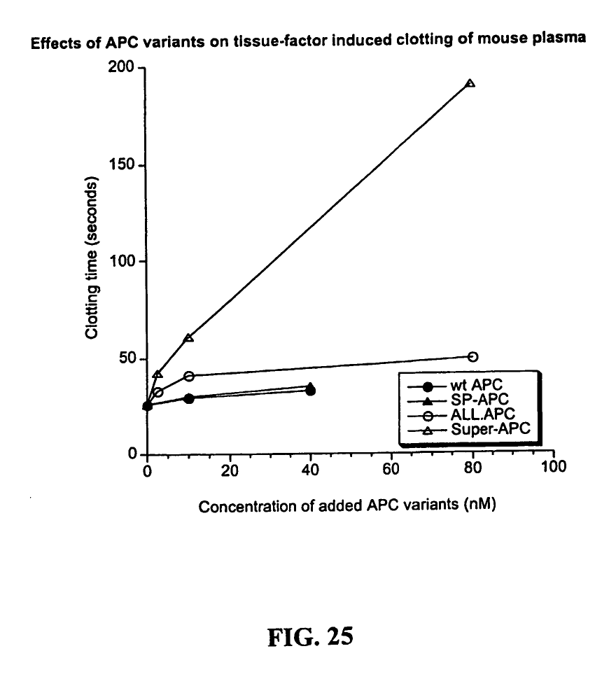

The results obtained in both APTT and tissue factor systems using mouse plasma were similar to those obtained with rat plasma (FIGS. 24 and 25). In both systems, the super-APC was most potent, ALL-APC being next in line when it relates to efficiency. In the APTT system, SP-APC was somewhat more potent than wt APC, whereas the two were similar in efficiency in the tissue factor based system. In particular, the tissue factor based system yielded very clear results with super-APC being superior to all other variants tested.

1. A variant blood coagulation component, which is substantially homologous in amino acid sequence to a wild-type blood coagulation component capable of exhibiting anticoagulant activity in the protein C-anticoagulant system of blood and selected from protein C(PC) and activated protein C (APC), said variant component being capable of exhibiting an anticoagulant activity, which is enhanced as compared to the anticoagulant activity expressed by the corresponding wild-type blood coagulation component, and said variant component differing from the respective wild-type component in that it contains in comparison with the said wild-type component at least one amino acid residue modification in its N-terminal amino acid residue sequence comprising the first 45 N-terminal amino acid residues and designated the Gla-domain, and at least one amino acid residue modification in a region of its amino acid residue sequence that corresponds to the serine-protease (SP) domain of the wild-type component.

2. The variant component of claim 1, which has at least 90% amino acid residue sequence identity with the corresponding wild-type component.

3. The variant component of claim 1, which has at least 95% amino acid residue sequence identity with the corresponding wild-type component.

4. The variant component of claim 1, which has at least 97% amino acid residue sequence identity with the corresponding wild-type component.

5. The variant component of any preceding claim, wherein the said at least one amino acid residue modification is comprised of a substituted, deleted or inserted amino acid residue.

6. The variant component of any preceding claim, wherein said component is a variant PC or a variant APC which exhibits enhanced membrane-binding affinity in comparison with the wild-type component.

7. The variant component of claim 6, which further exhibits enhanced calcium affinity as compared to wild-type protein C.

8. The variant component of any preceding claim, wherein the said variant component contains at least six, and optionally 7-10, amino acid residue modification in said Gla-domain.

9. The variant component of claim 1, wherein said variant component contains a modified Gla-domain, which contains the substitution mutations H10Q, S11G, S12N, D23S, Q32E, N33D and H44Y, said modified Gla-domain having the following amino acid sequence:
ANSFLEELRQ GNLERECIEE ICSFEEAKEI FEDVDDTLAF WSKYV (SEQ ID NO:5).

10. The variant component of any one of claims 1-7, wherein said Gla-domain contains an amino acid substitution at a position selected from positions 10, 11, 28, 32 or 33, and at least one further modification in the Gla-domain, optionally, said at least one further modification being selected from the positions 12, 23, or 44.

11. The variant component of any one of claims 1-7, wherein said at least one amino acid modificationin the Gla-domain is a substitution mutation at a position selected from positions 12, 23, and 44, said substitution mutation being selected from S12N, D23S and H44Y.

12. The variant component of any one of claims 1-7, wherein said at least one amino acid modification in the Gla-domain is located at a position selected from positions 10, 11, 12, 23, 32, 33 and 44 and, optionally, is a substitution mutation and wherein optionally all positions 10, 11, 12, 23, 32, 33 and 44 are modified.

13. The variant component of any preceding claim, wherein said component is a variant PC or a variant APC which exhibits enhanced proteolytic, suitably amidolytic, activity in comparison with the wild-type component.

14. The variant component of claim 1 which contains the same glycosylation sites as wild-type protein C, the amino acid residues at said sites being Asn.

15. The variant component of claim 1, wherein said at least one amino acid residue modification in the SP-domain is contained in a region corresponding to an amino acid stretch between amino acid residues numbers 290-320, suitably 300 and 314, of the wild-type component.

16. The variant component of claim 15, wherein the modified region, which corresponds to the wild-type amino acid residues numbers 300-314, contains the deletion Δ^{303, 304, 305, 308} and the substitution E307D/A310T and is represented by the formula WGYRDETKRNR (SEQ ID NO:7).

17. The variant component of claim 16, wherein said variant component contains a modified Gla-domain, which contains the substitution mutations H10Q, S11G, S12N, D23 S, Q32E, N33D and H44Y, said modified Gla-domain having the following amino acid sequence:
ANSFLEELRQ GNLERECIEE ICSFEEAKEI FEDVDDTLAF WSKYV (SEQ ID NO:5).

18. The variant component of any preceding claim, wherein said modification(s) in the Gla-domain is (are) substitutions.

19. The variant component of any preceding claim, that further contains at least one conservative substitution.

20. The variant component of any one of claims 1-19, wherein said wild-type blood coagulation component is of human origin.

21. A DNA segment comprising a nucleotide sequence coding for a variant blood coagulation component according to any preceding claim.

22. A recombinant DNA molecule comprising a replicable vector, which suitably is an expression vector, and a DNA segment according to claim 21 inserted therein.

23. A host cell comprising a microorganism or an animal cell, suitably a cultured animal cell line, harbouring the recombinant DNA molecule of claim 22, which suitably is stably incorporated therein.

24. The host cell of claim 23, which is an adenovirus-transfected human kidney cell.

25. A method for producing a DNA segment of claim 21 coding for a variant blood coagulation component according to any one of claims 1-20, which comprises:
(a) providing a DNA coding for the wild-type blood coagulation component;
(b) introducing nucleotide modifications in said wild-type DNA to form a modified DNA segment coding for said variant blood coagulation component; and
(c) replicating said modified DNA segment.

26. A method for producing a variant blood coagulation component according to any one of claims 1-20, which comprises:
(a) providing a DNA-segment that codes for said variant component;
(b) introducing said DNA segment provided in step (a) into an expression vector;
(c) introducing said vector, which contains said DNA segment, into a compatible host cell;
(d) culturing the host cell provided in step (c) under conditions required for expression of said variant component; and
(e) isolating the expressed variant component from the cultured host cell.

27. A pharmaceutical composition comprising an effective amount of a variant blood coagulation component according to any one of claims 1-20 and a pharmaceutically acceptable carrier, diluent or excipient.

28. A diagnostic test system, suitably in kit form, for assaying components participating in the protein C-anticoagulant system of blood, said system comprising a variant blood coagulation component of any one of claims 1-20.

29. The diagnostic test system of claim 28, wherein the variant blood coagulation component is a variant APC and said test system is a system for assaying functional activity of protein S or intact anticoagulant Factor V.

30. A method for inhibiting coagulation in a patient comprising administering to said patient a physiologically tolerable composition comprising a coagulation-inhibiting amount of a variant blood coagulation component according to any one of claims 1-20.

31. The method of claim 30, wherein thrombosis is inhibited.

32. The method of claim 31, wherein coagulation is inhibited in an individual having the blood coagulation disorder APC resistance.

33. Use of variant component of any one of claims 1-20 in the manufacture of a medicament for treatment or prevention of coagulation disorders, such as thrombosis.

34. Use according to claim 33, wherein the variant component comprises a variant PC or a variant APC in combination with a variant PS.

35. Use according to claim 33, in the manufacture of a medicament for treatment of APC resistance.

36. The variant component of claim 16, wherein the Gla-domain contains the mutations S11G, S12N, Q32E and N33D.

37. The variant component of claim 9, wherein said at least one modification in the SP-domain is a modification at position 302 or 316.


SEQUENCE LISTING (51 sequences)

SEQ ID NO: 1 - protein, 45 aa, Homo sapiens
ANSFLEELRHSSLERECIEEICDFEEAKEIFQNVDDTLAFWSKHV

SEQ ID NO: 2 - protein, 45 aa, Bovine sp.
ANSFLEELRPGNVERECSEEVCEFEEAREIFQNTEDTMAFWSKYS

SEQ ID NO: 3 - protein, 31 aa, Homo sapiens
QAGQETLVTGWGYHSSREKEAKRNRTFVLNF

SEQ ID NO: 4 - protein, 27 aa, Bovine sp.
QVGQETVVTGWGYRDETKRNRTFVLSF

SEQ ID NO: 5 - protein, 45 aa, Artificial Sequence
  Description of Artificial Sequence Partial modified human PC/APC sequence
ANSFLEELRQGNLERECIEEICSFEEAKEIFEDVDDTLAFWSKYV

SEQ ID NO: 6 - protein, 15 aa, Artificial Sequence
  Description of Artificial Sequence Partial modified human PC/APC sequence
WGYHSSREKEAKRNR

SEQ ID NO: 7 - protein, 11 aa, Artificial Sequence
  Description of Artificial Sequence Partial modified human PC/APC sequence
WGYRDETKRNR

SEQ ID NO: 8 - protein, 14 aa, Artificial Sequence
  Description of Artificial Sequence Partial modified human PC/APC sequence
WGYSSREKEAKRNR

SEQ ID NO: 9 - protein, 14 aa, Artificial Sequence
  Description of Artificial Sequence Partial modified human PC/APC sequence
WGYSSREKEAKRNR

SEQ ID NO: 10 - protein, 14 aa, Artificial Sequence
  Description of Artificial Sequence Partial modified human PC/APC sequence
WGYHSREKEAKRNR

SEQ ID NO: 11 - protein, 14 aa, Artificial Sequence
  Description of Artificial Sequence Partial modified human PC/APC sequence
WGYHSSEKEAKRNR

SEQ ID NO: 12 - protein, 14 aa, Artificial Sequence
  Description of Artificial Sequence Partial modified human PC/APC sequence
WGYHSSRKEAKRNR

SEQ ID NO: 13 - protein, 14 aa, Artificial Sequence
  Description of Artificial Sequence Partial modified human PC/APC sequence
WGYHSSREEAKRNR

SEQ ID NO: 14 - protein, 14 aa, Artificial Sequence
  Description of Artificial Sequence Partial modified human PC/APC sequence
WGYHSSREKAKRNR

SEQ ID NO: 15 - protein, 13 aa, Artificial Sequence
  Description of Artificial Sequence Partial modified human PC/APC sequence
WGYSREKEAKRNR

SEQ ID NO: 16 - protein, 13 aa, Artificial Sequence
  Description of Artificial Sequence Partial modified human PC/APC sequence
WGYHREKEAKRNR

SEQ ID NO: 17 - protein, 13 aa, Artificial Sequence
  Description of Artificial Sequence Partial modified human PC/APC sequence
WGYHSEKEAKRNR

SEQ ID NO: 18 - protein, 13 aa, Artificial Sequence
  Description of Artificial Sequence Partial modified human PC/APC sequence
WGYHSSKEAKRNR

SEQ ID NO: 19 - protein, 13 aa, Artificial Sequence
  Description of Artificial Sequence Partial modified human PC/APC sequence
WGYHSSREAKRNR

SEQ ID NO: 20 - protein, 13 aa, Artificial Sequence
  Description of Artificial Sequence Partial modified human PC/APC sequence
WGYHSSREAKRNR

SEQ ID NO: 21 - protein, 12 aa, Artificial Sequence
  Description of Artificial Sequence Partial modified human PC/APC sequence
WGYREKEAKRNR

SEQ ID NO: 22 - protein, 12 aa, Artificial Sequence
  Description of Artificial Sequence Partial modified human PC/APC sequence
WGYHEKEAKRNR

SEQ ID NO: 23 - protein, 12 aa, Artificial Sequence
  Description of Artificial Sequence Partial modified human PC/APC sequence
WGYHSKEAKRNR

SEQ ID NO: 24 - protein, 12 aa, Artificial Sequence
  Description of Artificial Sequence Partial modified human PC/APC sequence
WGYHSSEAKRNR

SEQ ID NO: 25 - protein, 12 aa, Artificial Sequence
  Description of Artificial Sequence Partial modified human PC/APC sequence
WGYHSSRAKRNR

SEQ ID NO: 26 - protein, 11 aa, Artificial Sequence
  Description of Artificial Sequence Partial modified human PC/APC sequence
WGYREEAKRNR

SEQ ID NO: 27 - protein, 11 aa, Artificial Sequence
  Description of Artificial Sequence Partial modified human PC/APC sequence
WGYREETKRNR

SEQ ID NO: 28 - protein, 11 aa, Artificial Sequence
  Description of Artificial Sequence Partial modified human PC/APC sequence
WGYHREAKRNR

SEQ ID NO: 29 - protein, 11 aa, Artificial Sequence
  Description of Artificial Sequence Partial modified human PC/APC sequence
WGYKEEAKRNR

SEQ ID NO: 30 - protein, 11 aa, Artificial Sequence
  Description of Artificial Sequence Partial modified human PC/APC sequence
WGYKDEAKRNR

SEQ ID NO: 31 - protein, 11 aa, Artificial Sequence
  Description of Artificial Sequence Partial modified human PC/APC sequence
WGYKEETKRNR

SEQ ID NO: 32 - protein, 11 aa, Artificial Sequence
  Description of Artificial Sequence Partial modified human PC/APC sequence
WGYRQETKRNR

SEQ ID NO: 33 - protein, 11 aa, Artificial Sequence
  Description of Artificial Sequence Partial modified human PC/APC sequence
WGYRQEAKRNR

SEQ ID NO: 34 - DNA, 36 bp, Artificial Sequence
  Description of Artificial Sequence oligonucleotide primer
aaattaatacgactcactatagggagacccaagctt

SEQ ID NO: 35 - DNA, 42 bp, Artificial Sequence
  Description of Artificial Sequence oligonucleotide primer
gtttctcttggtctcgtcacggtagccccagcccgtcacgag

SEQ ID NO: 36 - DNA, 36 bp, Artificial Sequence
  Description of Artificial Sequence oligonucleotide primer
cgtgacgagaccaagagaaaccgcaccttcgtcctc

SEQ ID NO: 37 - DNA, 36 bp, Artificial Sequence
  Description of Artificial Sequence oligonucleotide primer
gcatttaggtgacactatagaatagggccctctaga

SEQ ID NO: 38 - DNA, 42 bp, Artificial Sequence
  Description of Artificial Sequence oligonucleotide primer
ggcctccttctctcggctgctgtggtagccccagcccgtcac

SEQ ID NO: 39 - DNA, 41 bp, Artificial Sequence
  Description of Artificial Sequence oligonucleotide primer
cacagcagccgagagaaggaggccaagagaaaccgaccttc

SEQ ID NO: 40 - protein, 14 aa, Artificial Sequence
  Description of Artificial Sequence Partial modified protein C sequence
LVTGWGYRDETKRN

SEQ ID NO: 41 - protein, 12 aa, Artificial Sequence
  Description of Artificial Sequence Partial modified protein C sequence
RDETKRNRTFVL

SEQ ID NO: 42 - protein, 14 aa, Artificial Sequence
  Description of Artificial Sequence Partial modified protein C sequence
VTGWGYHSSREKEA

SEQ ID NO: 43 - protein, 4 aa, Artificial Sequence
  Description of Artificial Sequence Partial modified protein C sequence
RDET

SEQ ID NO: 44 - protein, 8 aa, Artificial Sequence
  Description of Artificial Sequence Partial modified protein C sequence
HSSREKEA

SEQ ID NO: 45 - protein, 14 aa, Artificial Sequence
  Description of Artificial Sequence Partial modified protein C sequence
HSSREKEAKRNRTF

SEQ ID NO: 46 - protein, 8 aa, Artificial Sequence
  Description of Artificial Sequence Partial modified protein C sequence
HSSREKEA

SEQ ID NO: 47 - DNA, 42 bp, Artificial Sequence
  Description of Artificial Sequence Oligonucleotide  primer
gcactcccgctccaggttgccttgacggagctcctccaggaa

SEQ ID NO: 48 - DNA, 33 bp, Artificial Sequence
  Description of Artificial Sequence Oligonucleotide  primer
atagaggagatctgtagcttcgaggaggccaag

SEQ ID NO: 49 - DNA, 33 bp, Artificial Sequence
  Description of Artificial Sequence Oligonucleotide  primer
cttggcctcctcgaagctacagatctcctctat

SEQ ID NO: 50 - DNA, 36 bp, Artificial Sequence
  Description of Artificial Sequence Oligonucleotide  primer
ctggtcaccgtcgacgtacttggaccagaaggccag

SEQ ID NO: 51 - DNA, 36 bp, Artificial Sequence
  Description of Artificial Sequence Oligonucleotide  primer
cagtgtgtcatccacatcttcgaaaatttccttggc